(19) United States
(12) Patent Application Publication
(10) Pub. No.: US 2023/0322952 A1
(43) Pub. Date: Oct. 12, 2023

(54) MULTISPECIFIC ANTIBODIES THAT BIND BOTH MAIT AND TUMOR CELLS

(71) Applicants: BIOMUNEX PHARMACEUTICALS, PARIS (FR); INSTITUT CURIE, PARIS CEDEX 05 (FR); INSERM (INSTITUT NATIONAL DE LA SANTE ET DE LA RECHERCHE MEDICALE), PARIS (FR)

(72) Inventors: OLIVIER LANTZ, PARIS (FR); SEBASTIAN AMIGORENA, PARIS (FR); MICHAEL SAITAKIS, PARIS (FR); MAUDE GUILLOT-DELOST, ORSAY (FR); EUGENE ZHUKOVSKY, BETHEL, CT (US); PIERRE-EMMANUEL GERARD, PARIS (FR); MUSTAPHA FAROUDI, SÈVRES (FR)

(21) Appl. No.: 17/791,549

(22) PCT Filed: Jan. 8, 2021
(86) PCT No.: PCT/EP2021/050253

§ 371 Date: Jul. 8, 2022

(30) Foreign Application Priority Data
Jan. 9, 2020 (EP) 20305011.7
Jan. 9, 2020 (EP) 20305012.5

(51) Int. Cl. C07K 16/46; A61P 35/00

(52) U.S. Cl. CPC C07K 16/468; A61P 35/00; C07K 2317/31; C07K 2317/55; C07K 2317/522; C07K 2317/565

(57) ABSTRACT

The invention provides a multispecific molecule capable of simultaneous binding to a Mucosal Associated Invariant T (MAIT) cell and a tumor cell, which multispecific molecule comprises at least one domain that specifically binds a Vα7.2 T cell receptor (TCR) and at least one domain that specifically binds a tumor associated antigen (TAA).

The present invention provides multispecific molecules useful for treating cancers.

### BACKGROUND OF THE INVENTION

T cell redirection approach using bispecific antibodies (BsAbs) has brought significant advancement for cancer immunotherapy. Two T cell redirecting BsAbs have received regulatory approval: catumaxomab for the treatment of malignant ascites and blinatumomab for acute lymphoblastic leukemia. Numerous others are undergoing clinical investigation. The accepted mechanism of action underlying T cell redirecting BsAb is via the formation of an immunological synapse (Offner et al, 2006; Nagorsen et al, 2011). This BsAb-mediated cross-linking of CD3 receptor and target cell Tumor Associated Antigen (TAA) results in: the activation of T cells, the subsequent release of perforin and granzyme from the cytotoxic granules into the milieu of the immunological synapse, and the ultimate destruction of the target cell by the ensuing apoptosis. In the case of the bispecific T cell engager (BiTE), the immunological synapses formed appear indistinguishable from those induced in the course of natural cytotoxic T cell recognition (Offner et al, 2006). Since delivery of these apoptotic mediators is accomplished by passive diffusion, the size of the synapse, defined by the distance between the anti-CD3 and anti-TAA moieties of the BsAb, are critical to cytotoxic potency. The distance between the TAA epitope and the target cell membrane determines the activity of the BiTE and may explain the differences in reported cytotoxic activity between different T cell redirecting BsAb formats, confirming that when the two cell membranes are in closest proximity the tumor cell lysis is most efficient.

Further, the activated T cells produce interleukin (IL)-2 and interferon (IFN)-γ that facilitates their proliferation and expansion at tumor sites, making T cells the most potent mediators of the immune response. CD8+ cells are the earliest to proliferate and exert their cytotoxic activity on target cells; however, CD4+ cells start with a short delay, but equally contribute to observed cytotoxicity.

Selection of optimal TAAs for employ with T cell redirecting mechanisms is difficult. Owing to the high cytotoxic potency of T cells, the therapeutic window of T cell redirecting approaches is rather narrow. Application to the treatment of solid tumors is difficult, mainly due to increased toxicity owing to broad expression of the selected tumor associated antigens in healthy cells and tissues (on-target off-tumor effects).

Current T cell redirecting BsAb target CD3, and thus will mobilize all CD3+ T cells at tumor sites including CD8+, which are the main effector cell population that mediates target cell killing, CD4+, which can elicit a cytokine storm, one of the main side-effects of this therapy, and undesirable Tregs, which when localized in target tissues reduce the immune response and suppress CD8+ effector cells by secreting immunosuppressive cytokines and activating inhibitory pathways on CTL (Koristka S, et al, 2012; Koristka et al, 2013). Several groups have reported that isolated Tregs can facilitate cytotoxic activity (Choi et al, 2013). However, it has also been demonstrated that the presence of Tregs facilitates in vivo tumor growth during treatment with a T cell redirecting BsAb targeting prostate stem cell antigen (PSCA/CD3) in a xenograft model (Koristka et al, 2012). Though one report indicates no proliferation of Tregs is observed in human ex vivo studies of a CD33/CD3 T cell redirecting BsAb (Krupka et al, 2014), exclusive redirection of CTLs may provide therapeutic benefit and further enhance the clinical efficacy of this class of drugs. To this end, a study (Michalk et al, 2014) demonstrated that a PSCA/CD8 BiTE molecule is capable of eliciting a potent anti-tumor response, albeit only pre-activated CD8+ T cells exhibited cytotoxicity.

Therefore, a new approach for more efficient and safer T cell redirection is required.

### SUMMARY OF THE INVENTION

The invention provides a T cell redirection approach that targets immune cells with invariant/semi-invariant T cell receptor (TCR) such as Mucosal Associated Invariant T (MAIT) cells and redirects these specific T cell to kill tumor cells.

More particularly, the invention provides a multispecific molecule capable of simultaneous binding to a MAIT cell and to a tumor cell, which multispecific molecule comprises at least one anti-Vα7.2 domain, i.e a domain that specifically binds a Vα7.2 TCR, and at least one anti-tumor associated antigen domain (TAA), i.e. a domain that specifically binds a TAA.

According to the invention, the crosslinking of the T cell receptor by means of such multispecific molecule activates the MAIT cells to kill tumor cells. See FIG. 1.

This approach has the advantages of: a) activating only cytotoxic cells against the target cells, b) not activating CD4+ T cells, hence less risk for a cytokine storm and autoreactivity, and c) not redirecting Tregs to the tumor site. Furthermore, since MAIT cells are abundant within human peripheral tissues, particularly in the liver and mucosal tissues, such as lung and gut, migration to solid tumors is favored.

The molecule is preferably a multispecific, preferably bispecific, antibody or an antigen-binding fragment thereof.

### LEGENDS TO THE FIGURES

FIG. 1 is a schematic drawing that shows how a bispecific antibody according to the invention targets a TAA at the tumor cell surface and the invariant TCR, namely the α chain Vα7.2.

FIG. 2 is a schematic drawing of an example of antibodies of the invention.

FIG. 3A shows the binding profile of anti-Vα7.2/anti-CD19 Fab-Fab on CD19⁺ Raji cells representative of four independent experiments.

FIG. 3B shows the binding profile of anti-Vα7.2/anti-CD19 Fab-Fab on Vα7.2⁺ cells representative of three different donors.

FIG. 4A shows the flow cytometry gating strategy for determining CD8⁺ T cell activation.

FIG. 4B shows the percentage of CD25⁺, CD69⁺ and double positive (CD²⁵⁺CD69⁺) CD8⁺TCRγδ⁻ T cells in the wells coated with different molar concentrations of either anti-Vα7.2/anti-CD19 Fab-Fab, or anti-CD3, or anti-Vα7.2, representative of two donors.

FIG. 5A shows the flow cytometry gating strategy for determining MAIT cells activation.

FIG. 5B shows the percentage of CD25⁺, CD69⁺ and double positive (CD²⁵⁺CD69⁺) MAIT (CD8⁺TCRδδ⁻CD161^{hi}IL18RA⁺) cells in the wells coated with different molar concentrations of either anti-Vα7.2/anti-CD19 Fab-Fab, or anti-CD3 or anti-Vα7.2, representative of two donors.

FIG. 6 shows the % specific lysis of Raji cells when co-cultured for 48 h at different concentrations of anti-Vα7.2/anti-CD19 Fab-Fab and different effector:target ratios.

FIG. 7A shows the binding profile of anti-Vα7.2/anti-CD19 Fab-Fab and anti-CD19/anti-Vα7.2 Fab-Fab antibodies, as well as that of a negative control Fab-Fab antibody on CD19+ NALM-6 tumor cells. Median of 3 independent experiments is shown.

FIG. 7B shows the binding profile of anti-Vα7.2/anti-CD19 BiXAb and anti-CD19/anti-Vα7.2 BiXAb antibodies, as well as that of a negative control BiXAb antibody on CD19+ NALM-6 tumor cells. Median of 3 independent experiments is shown.

FIG. 8 shows the flow cytometry gating strategy for determining MAIT cell binding within CD8+ enriched cells. One representative experiment is shown for Vα7.2/CD19 BiXAb antibody.

FIG. 9 shows the binding profile of anti-Vα7.2/anti-CD19 BiXAb and anti-CD19/anti-Vα7.2 BiXAb antibodies, as well as that of a negative control BiXAb antibody on Vα7.2+ CD8+ MAIT cells. Median of 3 independent experiments is shown.

FIG. 10 shows the percentages of CD69+ MAIT cells in the wells coated with anti-Vα7.2/anti-CD19 BiXAb or anti-CD19/anti-Vα7.2 BiXAb antibodies, or a negative control BiXAb antibody. One representative experiment of 2 independent experiments is shown.

FIG. 11 is a schematic drawing of cytotoxic assay.

FIG. 12 shows the percentages of CD69+ MAIT cells during cytotoxic assay with enriched CD8 T cells and A-549 tumor cells in the presence of anti-Vα7.2/anti-CD19 BiXAb or anti-CD19/anti-Vα7.2 BiXAb antibodies, or a negative control BiXAb antibody. One representative experiment of 2 independent experiments is shown.

FIG. 13 shows the specific lysis percentage of A-549 tumor cells when co-cultured for 48 h with CD8+ T cells in assay media containing rhIL-12 in the presence of anti-Vα7.2/anti-CD19 Fab-Fab or anti-CD19/anti-Vα7.2 Fab-Fab antibodies or negative control Fab-Fab antibody. The assay was performed at a 6:1 effector:target ratio. One representative experiment of 2 independent experiments is shown.

FIG. 14A shows the binding profile of anti-Her2/anti-Vα7.2 Fab-Fab antibodies, as well as that of a negative control Fab-Fab antibody on Her2+ A-549 tumor cells. Median of 3 independent experiments is shown.

FIG. 14B shows the binding profile of anti-Vα7.2/anti-Her2 BiXAb and anti-Her2/anti-Vα7.2 BiXAb antibodies, as well as that of a negative control BiXAb antibody on Her2+ A-549 tumor cells. Median of 3 independent experiments is shown.

FIG. 15 shows the binding profile of anti-Vα7.2/anti-Her2 BiXAb and anti-Her2/anti-Vα7.2 BiXAb antibodies, as well as that of a negative control BiXAb antibody on Vα7.2+ CD8+ MAIT cells. Median of 3 independent experiments is shown.

FIG. 16A shows the percentages of double positive CD69+CD25+ MAIT cells during cytotoxic assay with A-549 tumor cells and anti-Vα7.2/anti-Her2 Fab-Fab or anti-Her2/anti-Vα7.2 Fab-Fab antibodies, or a negative control Fab-Fab antibody. One representative experiment of 3 independent experiments is shown.

FIG. 16B shows the percentages of CD69+ MAIT cells during cytotoxic assay with A-549 tumor cells and anti-Vα7.2/anti-Her2 BiXAb or anti-Her2/anti-Vα7.2 BiXAb antibodies, or a negative control BiXAb antibody. One representative experiment of 3 independent experiments is shown.

FIG. 17A shows the specific lysis percentage of A-549 tumor cells when co-cultured for 48 h with CD8+ T cells in assay media containing rhIL-12 in the presence of anti-Vα7.2/anti-Her2 Fab-Fab or anti-Her2/anti-Vα7.2 Fab-Fab antibodies or a negative control Fab-Fab antibody. The assay was performed at a 6:1 effector:target ratio. One representative experiment of 3 independent experiments is shown.

FIG. 17B shows the specific lysis percentage of A-549 tumor cells when co-cultured for 48h with CD8+ T cells in assay media containing rhIL-12 in the presence of anti-Vα7.2/anti-Her2 BiXAb or anti-Her2/anti-Vα7.2 BiXAb antibodies or a negative control BiXAb antibody. The assay was performed at a 6:1 effector:target ratio. One representative experiment of 3 independent experiments is shown.

FIG. 18 shows the binding profile of anti-Vα7.2/anti-EGFR BiXAb, anti-EGFR/anti-Vα7.2 BiXAb antibodies, as well as that of a negative control BiXAb antibody on EGFR+ A-549 tumor cells. Median of 2 independent experiments is shown.

FIG. 19 shows the binding profile of anti-Vα7.2/anti-EGFR BiXAb, anti-EGFR/anti-Vα7.2 BiXAb antibodies, as well as that of a negative control BiXAb antibody on Vα7.2+ CD8+ MAIT cells. Median of 2 independent experiments is shown.

FIG. 20 is a schematic drawing of the in vivo experimental plan.

FIG. 21A shows the in vivo efficacy of the anti-Vα7.2/anti-CD19 Fab-Fab or anti-anti-Vα7.2/anti-HER2 Fab-Fab antibodies in NSG mice; the animals were inoculated with the A-549/luciferase tumor cell line expressing HER2 and CD19 on day 0 and subsequently with the PBMC on days 1 and 4. The data are reported as average bioluminescence signal from each mouse.

FIG. 21B shows the in vivo efficacy of the anti-Vα7.2/anti-CD19 BiXAb or anti-Vα7.2/anti-HER2 BiXAb antibodies in NSG mice; the animals were inoculated on day 0 with the A-549/luciferase tumor cell line expressing HER2 and CD19 and subsequently with the PBMC on days 1 and 4. The data are reported as average bioluminescence signal from each mouse.

### DETAILED DESCRIPTION OF THE INVENTION

### Definitions

The basic structure of a naturally occurring antibody molecule is a Y-shaped tetrameric quaternary structure consisting of two identical heavy chains and two identical light chains, held together by non-covalent interactions and by inter-chain disulfide bonds.

In mammalian species, there are five types of heavy chains: α, δ, ϵ, γ, and μ, which determine the class (isotype) of immunoglobulin: IgA, IgD, IgE, IgG, and IgM, respectively. The heavy chain N-terminal variable domain (VH) is followed by a constant region, containing three domains (numbered CH1, CH2, and CH3 from the N-terminus to the C-terminus) in γ, α, and δ heavy chains, while the constant regions of p and c heavy chains are composed of four domains (numbered CH1 , CH2, CH3 and CH4 from the N-terminus to the C-terminus). The CH1 and CH2 domains of IgA, IgG, and IgD are separated by a flexible hinge, which varies in length between the different classes and in the case of IgA and IgG, between the different subtypes: IgG1, IgG2, IgG3, and IgG4 have respectively hinges of 15, 12, 62 (or 77), and 12 amino acids, and IgA1 and IgA2 have respectively hinges of 20 and 7 amino acids.

There are two types of light chains: A and K, which can associate with any of the heavy chain isotypes, but are both of the same type in a given antibody molecule. Both light chains appear to be functionally identical. Their N-terminal variable domain (VL) is followed by a constant region consisting of a single domain termed CL.

The heavy and light chains pair by protein/protein interactions between the CH1 and CL domains, and between the VH and VL domains, and the two heavy chains associate by protein/protein interactions between their CH3 domains.

The antigen-binding regions correspond to the arms of the Y-shaped structure, which consist each of the complete light chain paired with the VH and CH1 domains of the heavy chain, and are called the Fab fragments (for Fragment antigen binding). Fab fragments were first generated from native immunoglobulin molecules by papain digestion which cleaves the antibody molecule in the hinge region, on the amino-terminal side of the interchain disulfide bonds, thus releasing two identical antigen-binding arms. Other proteases such as pepsin, also cleave the antibody molecule in the hinge region, but on the carboxy-terminal side of the interchain disulfide bonds, releasing fragments consisting of two identical Fab fragments and remaining linked through disulfide bonds; reduction of disulfide bonds in the F(ab′)2 fragments generates Fab′ fragments.

The part of the antigen-binding region corresponding to the VH and VL domains is called the Fv fragment (for Fragment variable); it contains the CDRs (complementarity determining regions), which form the antigen-binding site (also termed paratope).

The effector region of the antibody which is responsible for its binding to effector molecules on immune cells, corresponds to the stem of the Y-shaped structure, and contains the paired CH2 and CH3 domains of the heavy chain (or the CH2, CH3 and CH4 domains, depending on the class of antibody), and is called the Fc (for Fragment crystallisable) region.

Due to the identity of the two heavy chains and the two light chains, naturally occurring antibody molecules have two identical antigen-binding sites and thus bind simultaneously to two identical epitopes.

In the context of the invention, the “multispecific antigen-binding fragment” is defined herein as a molecule having two or more antigen-binding regions, each recognizing a different epitope. The different epitopes can be borne by a same antigenic molecule or by different antigenic molecules. The term “recognizing” or “recognizes” means that the fragment specifically binds a target antigen.

An antibody “specifically binds” to a target antigen if it binds with greater affinity, avidity, more readily, and/or with greater duration than it binds to other substances. “Specific binding” or “preferential binding” does not necessarily require (although it can include) exclusive binding. Generally, but not necessarily, reference to binding means preferential binding. Preferably the molecule will not show any significant binding to ligands other than its specific target (e.g., an affinity of about 100-fold less), i.e. minimal cross-reactivity.

“Affinity” is defined as the strength of the binding interaction of two molecules, such as an antigen and its antibody, which is defined for antibodies and other molecules with more than one binding site as the strength of binding of the ligand at one specified binding site. Although the noncovalent attachment of a ligand to antibody is typically not as strong as a covalent attachment, “High affinity” is for a ligand that binds to an antibody having an affinity constant (Ka) of about 10⁶ to 10¹¹ M⁻¹.

The terms “subject,” “individual,” and “patient” are used interchangeably herein and refer to a mammal being assessed for treatment and/or being treated. Subjects may be human, but also include other mammals, particularly those mammals useful as laboratory models for human disease, e.g. mouse, rat, rabbit, dog, etc.

The term “treatment” or “treating” refers to an action, application or therapy, wherein a subject, including a human being, is subjected to medical aid with the purpose of improving the subject's condition, directly or indirectly. Particularly, the term refers to reducing incidence, or alleviating symptoms, eliminating recurrence, preventing recurrence, preventing incidence, improving symptoms, improving prognosis or combination thereof in some embodiments. The skilled artisan would understand that treatment does not necessarily result in the complete absence or removal of symptoms. For example, with respect to cancer, “treatment” or “treating” may refer to slowing neoplastic or malignant cell growth, proliferation, or metastasis, preventing or delaying the development of neoplastic or malignant cell growth, proliferation, or metastasis, or some combination thereof.

Mucosal associated invariant T (MAIT) cells are non-conventional T cells that are not restricted by classical MHC and are found in blood and tissues, where they contribute to barrier immunity. They have the potential to redirect cytotoxicity based upon expression studies (Salou et al, 2019) and in vitro assays (Le Bourhis et al, 2013). MAIT cells express a semi-invariant TCR (named Vα7.2) which recognizes Vitamin B2 precursors presented by the highly evolutionarily conserved MHC class Ib molecule, MR1 (Franciszkiewicz et al, 2016; Salou et al, 2017). This receptor is also designated TRAV1/TRAJ according to the WHO-IUIS nomenclature for T-cell receptor (TCR) gene segments of the immune system, reported in Bull World Health Organ. 1993; 71(1): 113-115.

The MAIT cells represent around 1 to 10% of T cells in blood, but also reside in tissues and organs such as lung, liver, skin and the colon. Moreover, MAIT cells can have cytotoxic activity and produce IFNγ and TNFα upon activation (Dusseaux et al, 2011).

Vα7.2 is the alpha chain of the T cell receptors expressed by MAIT cells. The term includes Vα7.2-Jα33, Vα7.2-Jα20 or α7.2-Jα12 alpha chains. In humans, they consist of TRAV1-2 joined to TRAJ33, TRAJ20 or TRAJ12 with little to no n nucleotide additions at the TCR-α complementarity determining region 3 (CDR3α) junction. As used herein, “Vα7.2-Jα33/20/12” includes any variant, derivative, or isoform of the rearranged Vα7.2-Jα33/20/12 gene or encoded protein. The amino acid sequence of human and mouse Vα7.2-Jα33 are described in Tilloy et al, 1999 while Vα7.2-Jα20 and Vα7.2-Jα12 are described in Reantragoon et al, 2013. Sequence of human Vα7.2-Jα33 is shown as SEQ ID NO:1. Sequence of Vα7.2-Jα12 is shown as SEQ ID NO:2, and Jα20 as SEQ ID NO:3.

The term “cancer” refers to a disease characterized by the uncontrolled (and often rapid) growth of aberrant cells. Cancer cells can spread locally or through the bloodstream and lymphatic system to other parts of the body.

The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

As used herein, the term “tumor-associated antigen” or “TAA” refers to a molecule (typically a protein, carbohydrate, lipid or some combination thereof) that is expressed (or overexpressed relative to normal tissues) on the surface of a cancerous cell, either entirely or as a fragment (e.g., MHC/peptide). As used herein, the term “cancerous cell” refers to a cell that is undergoing or has undergone uncontrolled proliferation. In some embodiments, a TAA is a marker expressed by both normal cells and cancer cells, e.g., CD19, as described in greater details below. In some embodiments, a TAA is a cell surface molecule that is overexpressed in a cancerous cell in comparison to a normal cell, for instance, 2-fold overexpression, 3-fold overexpression or more in comparison to a normal cell/tissue. In some embodiments, a TAA is a cell surface molecule that is inappropriately synthesized in the cancerous cell, for instance, a molecule that contains deletions, additions or mutations (e.g. EGFRvIII) in comparison to the molecule expressed on a normal cell. In some embodiments, a TAA will be expressed exclusively on the cell surface of a cancerous cell, entirely or as a fragment (e.g., MHC/peptide), and not synthesized or expressed on the surface of a normal cell. Accordingly, the term “TAA” encompasses cell antigens that are specific to cancer cells, sometimes known in the art as tumor-specific antigens (“TSAs”).

### The Anti-Vα7.2 Domain

The multispecific molecules of the invention comprise at least one domain that binds Vα7.2, e.g. Vα7.2-Jα33, Vα7.2-Jα20 and/or Vα7.2-Jα12.

Such binding domain can derive from any anti-Vα7.2 antibody. Methods for producing such antibodies are known in the art. Examples of such antibodies are disclosed in international patent application WO2008/087219.

It will be appreciated that the multispecific molecules of the invention can recognize any part of the Vα7.2-Jα33, Vα7.2-Jα20 and/or Vα7.2-Jα12 polypeptide, e.g. Vα7.2-Jα33/Vβ2 or Vα7.2-Jα33/Vβ2 polypeptide. For example, Voc7, Voc7.2, Joc33, fragments thereof, or any combination of any of these polypeptides or fragments, can be used as immunogens to raise antibodies, and the antibodies of the invention can recognize epitopes at any location within the Vα7.2-Joc33 (or, e.g., Vα7.2-Jα33/Vβ2 or Vα7.2-Jα33/Vβ2) polypeptide. Preferably, the recognized epitopes are present on the cell surface, i.e. they are accessible to antibodies present outside of the cell.

In a particular embodiment, the domain that binds Vα7.2 is an antigen-binding fragment from an anti- Vα7.2 antibody that is capable of competing or binds to the same or substantially the same epitope of the Vα7.2-Jα33 polypeptide as monoclonal antibody 3C10 described in international patent application WO2008/087219. When an antibody or agent is said to “compete” or “bind to substantially the same epitope” as a particular monoclonal antibody (e. g. 3C10), it means that the antibody or agent competes with the monoclonal antibody in a binding assay using either recombinant Vα7.2-Joc33 molecules or surface expressed Vα7.2-Joc33 molecules. For example, if a test antibody or agent reduces the binding of 3C10 to a Vα7.2-Joc33 polypeptide in a binding assay, the antibody or agent is said to “compete” with 3C10 or 1A6, respectively.

In a particular embodiment, the multispecific molecules of the invention comprises a heavy variable chain that comprises the following CDRs: GFNIKDTH (SEQ ID NO: 4); TDPASGDT (SEQ ID NO:5) and CAHYYRDDVNYAMDY (SEQ ID NO:6); and/or a light variable chain that comprises the following CDRs: QNVGSN (SEQ ID NO:7); SSS, and QQYNTYPYT (SEQ ID NO:8) of the 3C10 antibody.

### The Anti-TAA Domain

The multispecific molecules of the invention comprise at least one domain that binds a TAA. Particular examples of such TAAs include CD19, CD20, CD38, EGFR, HER2, VEGF, CD52, CD33, RANK-L, GD2, CD33, CEA family (including CEACAM antigens, e.g. CEACAM1, CEACAM5; or PSG antigen), MUC1, PSCA, PSMA, GPA33, CA9, PRAME, CLDN1, HER3, and glypican-3, as well as CD22, CD25, CD40, CD30, CD79b, CD138 (syndecan-1), BCMA, SLAMF7 (CS1, CD319), CD56, CCR4, EpCAM, PDGFR-α, Apo2L/TRAIL, PD-L1. CD19, EGFR, HER2 are particularly preferred.

Such multispecific antibodies, that bind CD19, EGFR, or HER2, are described in greater details below.

Generally speaking, any person skilled in the art knows how to produce antibodies that specifically bind to any of said TAAs. Many are commercialized.

In preferred embodiments, the multispecific molecules of the invention comprise humanized or chimeric antigen-binding fragments.

### Design of the Multispecific Antibodies

It is herein provided multispecific antigen-binding fragment(s) and multispecific antibody constructs, comprising said fragments, wherein each multispecific antigen-binding fragment consists essentially of tandemly arranged Fab fragments.

Such fragments and constructs preferably comprise chains from human immunoglobulins, preferably IgG, still preferably IgG1.

In case of a multispecific antigen-binding fragment comprising more than two different Fab fragments, the polypeptide linkers separating the Fab fragments can be identical or different.

According to a preferred embodiment, it is provided a multispecific antibody that comprises two identical antigen-binding arms, each consisting of a multispecific antigen-binding fragment as defined above. The antigen-binding arms can be linked together in diverse ways.

If one wishes to obtain an antibody without Fc-mediated effects or an antibody monovalent for each of the two antigens it targets, the antibody will comprise no Fc region. In this case, the two antigen-binding arms can be linked together for instance:
by homodimerization of the antigen-binding arms through the inter-chain disulfide bonds provided by the polypeptide linker(s) separating the Fab fragments; and/or
through the addition at the C-terminal end of each antigen-binding arm, of a polypeptide extension containing cysteine residues allowing the formation of inter-chain disulfide bonds, and homodimerization of said polypeptide extension resulting in a hinge-like structure; by way of non-limiting examples, said polypeptide extension may be for instance a hinge sequence of an IgG1, IgG2 or IgG3;
through a linker, preferably a semi-rigid linker, joining the C-terminal ends of the heavy chains of the two antigen-binding arms to form a single polypeptide chain and maintaining said antigen-binding arms at a sufficient distance between each other.

Alternatively, if effector functions such as antibody-dependent cell-mediated cytotoxicity (ADCC), complement-dependent cytotoxicity (CDC) and/or antibody-dependent phagocytosis (ADP) or bivalent binding for each of the two antigens are desired, a multispecific antibody of the invention can further comprise a Fc domain providing these effector functions. The choice of the Fc domain will depend on the type of desired effector functions.

In this case, a multispecific antibody of the invention has an immunoglobulin-like structure, comprising:
two identical multispecific antigen-binding arms as defined above;
the dimerized CH2 and CH3 domains of an immunoglobulin;
either the hinge region of an IgA, IgG, or IgD, linking the C-terminal ends of the CH1 domains of the antigen-binding arms to the N-terminal ends of the CH2 domains, or alternatively, when the CH4 domains that follow the CH3 domains come from an IgM or IgE, the C-terminal ends of the CH1 domains of the antigen-binding arms in this case can be linked directly to the N-terminal ends of the CH2 domains.

Preferably, the CH2 and CH3 domains, the hinge region and/or the CH4 domains are derived from a same immunoglobulin or from immunoglobulins of the same isotype and subclass as the CH1 domains of the antigen-binding arm.

The CH2, CH3, and optionally CH4 domains, as well as the hinge regions from native immunoglobulins can be used. It is also possible to mutate them, if desired, for instance in order to modulate the effector function of the antibody. In some instances, whole or part of the CH2 or the CH3 domain can be omitted.

The invention more particularly provides bispecific tetravalent antibodies, comprising two binding sites to each of their targets, and a functional Fc domain allowing the activation of effector functions such as antibody-dependent cell-mediated cytotoxicity (ADCC) and phagocytosis. Such preferred antibodies are full length antibodies. However preferred antibodies carry mutations in the Fc domain so as to avoid or reduce binding to Fc gamma receptors.

The antibodies preferably comprise heavy chains and light chains from human immunoglobulins, preferably IgG, still preferably IgG1.

The light chains may be lambda or kappa light chains; they preferably are Kappa light chains.

In a preferred embodiment, a linker links IgG Fab domains in a tetra-Fab bispecific antibody format, the amino acid sequence of which comprises the heavy chain sequences of at least two Fab domains joined by said polypeptide linker, followed by the native hinge sequence, followed by the IgG Fc sequence, co-expressed with the appropriate IgG light chain sequences.

An example of the antibodies of the invention, named BiXAb antibodies, which have an IgG-like structure, is illustrated in FIG. 2.

In a particular embodiment, the bispecific antibodies of the invention comprise
a continuous heavy chain constructed of an Fc (Hinge-CH2-CH3)
followed by antibody 1 Fab heavy chain (CH1-VH) and the successive Fab heavy chain (CH1-VH) of antibody 2, the latter joined by a polypeptide linker sequence, e.g. a linker as described in greater details below,
and during protein expression the resulting heavy chain assembles into dimers while the co-expressed antibody 1 and antibody 2 light chains (VL-CL) associate with their cognate heavy chains to form the final tandem F(ab)′2-Fc molecule,
   the antibody 1 (Ab1) and the antibody 2 (Ab2) being different.

In a preferred embodiment, described are bispecific antibodies, which comprise two Fab fragments with different CH1 and CL domains consisting of
a) Fab fragment having CH1 and C-Kappa domains derived from a human IgG1/Kappa, and the VH and VL domains of Ab1,
b) Fab fragment having CH1 and C-Kappa domains derived from a human IgG1/Kappa and the VH and VL domains of Ab2,
c) a mutated light chain CL constant domain which is derived from human Kappa constant domain,
d) a mutated heavy chain CH1 constant domain

the Fab fragments being tandemly arranged in the following order
the C-terminal end of the CH1 domain of Ab1 Fab fragment being linked to the N-terminal end of the VH domain of Ab2 Fab fragment through a polypeptide linker,
the hinge region of a human IgG1 linking the C-terminal ends of CH1 domain of Ab2 fragment to the N-terminal of the CH2 domain,
the dimerized CH2 and CH3 domains of a human IgG1, preferably with one or several mutations that reduce or eliminate the interaction with Fc gamma receptors.

According to the invention, Ab1 and Ab2 are, independently, an antibody that specifically binds Vα7.2 (such as those described in greater details above), and an antibody that specifically binds a tumor associated antigen, or vice versa.

A preferred construct of the invention is a multispecific antigen-binding fragment Fab-Fab, which does not contain the Fc domain. A particular Fab-Fab construct according to the invention is described in Example 1.

Such Fab-Fab constructs typically comprise two different Fab domains. They possess the same Light Chains as in the corresponding BiXAb antibodies; however, the Heavy Chain of Fab-Fabs is shortened in such a fashion so that their most C-terminal residue is Cysteine-220 (in EU numbering).

The assembly of Fab domains is accomplished via natural pairing of Light and Heavy chains without the use of peptide linkers.

In order to maximize propensity of cognate pairing between Light and Heavy chains, one may contemplate introducing mutations at the interface of Light and Heavy chains (CL/CH1 interface) in Fab fragments.

In preferred embodiments, each CH1 domain carries at least one mutation, and each CL1 domain also carries at least one mutation, which mutations are selected so that a correct cognate pairing of the CH1 and CL1 domains is improved.

These mutations can be selected from the following list:
de novo-introduced ionic pairs or reversed polarity charged mutations of native ionic pairs already present at the interface of the Heavy and Light chains of the Fab fragment;
“knobs-into-holes” mutations;
mutations that resurface opposing constant regions of Heavy and Light chain interfaces in Fab fragments to change them from strongly polar to highly hydrophobic or vice versa.

Several sets of mutations are thus suitable, as described in greater details below.

Of note, throughout the present description, amino acid sequences and the sequence position numbers used herein for the CH1 and CL domains are defined according to Kabat et al, Sequences of Proteins of Immunological Interest, 5th Ed. Public Health Service, National Institutes of Health, Bethesda, Md. (1991).

Residues that can be mutated in the VL domain may be e.g. selected from the group consisting of D 1, W 36, Q 38, A 43, P 44, T 85, F 98, and Q 100 (e.g. Q100C).

Residues that can be mutated in the CL kappa domain may be e.g. selected from the group consisting of S 114, F 116, F 118, E 123 (e.g. E123K), Q 124, T 129, S 131, V 133, L 135, N 137, Q 160, S 162, S 174, S 176, T 178, and T 180.

Residues that can be mutated in the CL lambda domain may be e.g. selected from the group consisting of S 114, T 116, F 118, E 123, E 124, K 129, T 131, V 133, L 135, S 137, V 160, T 162, A 174, S 176, Y 178, and S 180.

Residues that can be mutated in the VH domain may be e.g. selected from the group consisting of V 37, Q 39, G 44 (e.g. G44C), R 62, F 100, W 103, and Q 105.

Residues that can be mutated in the CH1 domain may be e.g. selected from the group consisting of L 124, A 139, L 143, D 144, K 145, D 146, H 172, F 174, P 175, Q 179, S 188, V 190, T 192, and K 221 (e.g. K221E).

Specific mutations are described in US patent applications 2014/0200331, 2014/150973, 2014/0154254, and international patent application WO2007/147901, all incorporated herein by reference.

In a preferred embodiment, a pair of interacting polar interface residues is exchanged for a pair of neutral and salt bridge forming residues. The replacement of Thr192 by a glutamic acid or aspartic acid on CH1 chain and exchange of Asn137 to a Lys on CL chain can be selected, optionally with a substitution of the serine residue at position 114 of said CL domain with an alanine residue.

In another set of mutations, one can replace the Leu143 of the CHI domain by a Gin residue, while the facing residue of the CL chain, that is Vα133, is replaced by a Thr residue. This first double mutation constitutes the switch from hydrophobic to polar interactions. Simultaneously a mutation of two interacting serines (Ser188 on CH1 chain and Ser176 on CL chain) to valine residues can achieve a switch from polar to hydrophobic interactions.

In yet another embodiment, the mutations can comprise substitution of the leucine residue at position 124 of CH1 domain with a glutamine and substitution of the serine residue at position 188 of CH1 domain with a valine residue; and substitution of the valine residue at position 133 of CL domain with a threonine residue and substitution of the serine residue at position 176 of said CL domain with a valine residue.

The “knob into holes” mutations include a set of mutations (KH1) wherein Leu 124 and Leu 143 of the CH1 domain have been respectively replaced by an Ala and a Glu residue while the Val 33 of the CL chain has been replaced by a Trp residue, while, in the set of mutations named H2, the Val 90 of the CH1 domain has been replaced by an Ala residue, and the Leu135 and Asn137 of the CL chain have respectively been replaced by a Trp and an Ala residue.

The preferred mutations are disclosed below:

TABLE 1

| Preferred mutations | Name of the |
|---|---|
| (LC is Light Chain, HC is Heavy Chain) | mutation set |
| LC(S114A/N137K), HC(T192E) | CR3 mutation |
| LC(S114A/N137K), HC(T192D) | CC1 mutation |
| LC(V133T/S176V), | Mut4 |
| HC(L143Q/S188V) | |
| LC(V133T/S176V), | ML1 |
| HC(L124Q/S188V) | |

In a particular embodiment, the multispecific antibody may carry a double mutation, e.g. one arm with the CR3 mutation and the other with the Mut4 mutation.

In a particular embodiment, the multispecific antibody further comprises a Fc region of an immunoglobulin comprising Hinge-CH2-CH3 domains, which Fc region is linked to both antigen-binding arms by said Hinge domain, linking the C-terminal ends of CH1 domains of the antigen-binding arms to the N-terminal ends of the CH2 domains. Specific mutations at the interface in the CH3 or CH2 domains of the Fc may be contemplated to favor hetero-dimerization of two heavy chains instead of their natural homo-dimerization. Such mutations may be selected from the following list:
de novo-introduced ionic pairs or reversed polarity charged mutations of native ionic pairs already present at the interface of two Heavy chains of the Fc domain;
knobs-into-holes types mutations, well known and described in the art;
mutations that resurface two opposing Heavy chain interfaces, e.g. to change them from strongly polar to highly hydrophobic, or vice versa.
   Also, specific mutations in the IgG1 Fc domain decreasing or eliminating binding to Fc gamma receptors may be utilized, including but not limited to:
L234A/L235A
N297A (eliminating N-linked glycosylation site)
L234A/L235A/G237A/P238S/H268A/A330S/P331S
Or specific combination of positional substitutions of any of the following residues: L234A, L235A, G236R, G237A, P238S, H268A, L328R, A330S, P331S (EU numbering)

Any of the molecules described herein can be modified to contain additional non-proteinaceous moieties that are known in the art and readily available, e.g., by PEGylation, hyperglycosylation, and the like. Modifications that can enhance serum half-life or stability against proteolytic degradation are of interest.

The antibodies of the invention may be glycosylated or not, or may show a variety of glycosylation profiles. In a preferred embodiment, antibodies are unglycosylated on the variable region of the heavy chains, but are glycosylated on the Fc region.

One may use humanized forms of a reference non-human antibody. In a humanization approach, complementarity determining regions (CDRs) and certain other amino acids from donor variable regions are grafted into human variable acceptor regions and then joined to human constant regions. See, e.g. Riechmann et al., Nature 332:323-327 (1988); U.S. Pat. No. 5,225,539.

### Design of the Linkers

In a particular embodiment, a polypeptide linker is used to link the Fab fragment that binds Vα7.2, and the Fab fragment that binds the tumor associated antigen.

It is also designated “hinge-derived polypeptide linker sequence” or “pseudo hinge linker”, and comprises all or part of the sequence of the hinge region of one or more immunoglobulin(s) selected among IgA, IgG, and IgD, preferably of human origin. Said polypeptide linker may comprise all or part of the sequence of the hinge region of only one immunoglobulin. In this case, said immunoglobulin may belong to the same isotype and subclass as the immunoglobulin from which the adjacent CH1 domain is derived, or to a different isotype or subclass. Alternatively, said polypeptide linker may comprise all or part of the sequences of hinge regions of at least two immunoglobulins of different isotypes or subclasses. In this case, the N-terminal portion of the polypeptide linker, which directly follows the CH1 domain, preferably consists of all or part of the hinge region of an immunoglobulin belonging to the same isotype and subclass as the immunoglobulin from which said CH1 domain is derived.

Optionally, said polypeptide linker may further comprise a sequence of from 2 to 15, preferably of from 5 to 10 N-terminal amino acids of the CH2 domain of an immunoglobulin.

The polypeptide linker sequence typically consists of less than 80 amino acids, preferably less than 60 amino acids, still preferably less than 40 amino acids.

In some cases, sequences from native hinge regions can be used; in other cases point mutations can be brought to these sequences, in particular the replacement of one or more cysteine residues in native IgG1, IgG2 or IgG3 hinge sequences by alanine or serine, in order to avoid unwanted intra-chain or inter-chains disulfide bonds.

In a particular embodiment, the polypeptide linker sequence comprises or consists of amino acid sequence EPKX1CDKX2HX3X4PPX5PAPELLGGPX6X7PPX8PX9PX1OGG (SEQ ID NO:9), wherein X1, X2, X3, X4, X5, X6, X7, X8, X9, X10, identical or different, are any amino acid. In particular, the polypeptide linker sequence may comprise or consist of a sequence selected from the group consisting of

(SEQ ID NO: 10)
EPKSCDKTHTSPPAPAPELLGGPGGPPGPGPGGG;
(SEQ ID NO: 11)
EPKSCDKTHTSPPAPAPELLGGPAAPPAPAPAGG;
(SEQ ID NO: 12)
EPKSCDKTHTSPPAPAPELLGGPAAPPGPAPGGG;
(SEQ ID NO:_13)
EPKSCDKTHTCPPCPAPELLGGPSTPPTPSPSGG
and
(SEQ ID NO: 14)
EPKSCDKTHTSPPSPAPELLGGPSTPPTPSPSGG.

In a particular embodiment, X1, X2 and X3, identical or different, are Threonine (T) or Serine (S)

In another particular embodiment, X1, X2 and X3, identical or different, are selected from the group consisting of Ala (A), Gly (G), Val (V), Asn (N), Asp (D) and Ile (I), still preferably X1, X2 and X3, identical or different, may be Ala (A) or Gly (G).

Alternatively, X1, X2 and X3, identical or different, may be Leu (L), Glu (E), Gln (Q), Met (M), Lys (K), Arg (R), Phe (F), Tyr (T), His (H), Trp (W), preferably Leu (L), Glu (E), or Gln (Q).

In a particular embodiment, X4 and X5, identical or different, are any amino acid selected from the group consisting of Serine (S), Cysteine (C), Alanine (A), and Glycine (G).

In a preferred embodiment, X4 is Serine (S) or Cysteine (C).

In a preferred aspect, X5 is Alanine (A) or Cysteine (C).

In a particular embodiment, X6, X7, X8, X9, X10, identical or different, are any amino acid other than Threonine (T) or Serine (S). Preferably X6, X7, X8, X9, X10, identical or different, are selected from the group consisting of Ala (A), Gly (G), Val (V), Asn (N), Asp (D) and Ile (I). Alternatively, X6, X7, X8, X9, X10, identical or different, may be Leu (L), Glu (E), Gln (Q), Met (M), Lys (K), Arg (R), Phe (F), Tyr (T), His (H), Trp (W), preferably Leu (L), Glu (E), or Gln (Q).

In a preferred embodiment, X6, X7, X8, X9, X10, identical or different, are selected from the group consisting of Ala (A) and Gly (G).

In still a preferred embodiment, X6 and X7 are identical and are preferably selected from the group consisting of Ala (A) and Gly (G).

In a preferred embodiment, the polypeptide linker sequence comprises or consists of sequence SEQ ID NO: 9, wherein
X1, X2 and X3, identical or different, are Threonine (T), Serine (S);
X4 is Serine (S) or Cysteine (C);
X5 is Alanine (A) or Cysteine (C);
X6, X7, X8, X9, X10, identical or different, are selected from the group consisting of Ala (A) and Gly (G).

In another preferred embodiment, the polypeptide linker sequence comprises or consists of sequence SEQ ID NO: 9, wherein
X1, X2 and X3, identical or different, are Ala (A) or Gly (G);
X4 is Serine (S) or Cysteine (C);
X5 is Alanine (A) or Cysteine (C);
X6, X7, X8, X9, X10, identical or different, are selected from the group consisting of Ala (A) and Gly (G).

In embodiments wherein the antibodies comprise different Fab fragments, the polypeptide linkers separating the Fab fragments can be identical or different.

### Production of the Multispecific Antibodies

Nucleic acids encoding heavy and light chains of the antibodies of the invention are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Such control sequences include a signal sequence, a promoter, an enhancer, and a transcription termination sequence. Expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences.

In one example, both the heavy and light chain coding sequences (e.g., sequences encoding a VH and a VL, a VH-CH1 or a VL-CL, are included in one expression vector. In another example, each of the heavy and light chains of the antibody is cloned into an individual vector. In the latter case, the expression vectors encoding the heavy and light chains can be co-transfected into one host cell for expression of both chains, which can be assembled to form intact antibodies either in vivo or in vitro.

In a particular embodiment, a host cell is co-transfected with three independent expression vectors, such as plasmids, leading to the coproduction of all three chains (namely the heavy chain HC, and two light chains LC1 and LC2, respectively) and to the secretion of the multispecific antibody.

More especially the three vectors may be advantageously used in a following molecular ratio of 3:2:2 (HC:LC1:LC2).

The recombinant vectors for expression of the antibodies described herein typically contain a nucleic acid encoding the antibody amino acid sequences operably linked to a promoter, either constitutive or inducible. The vectors can be suitable for replication and integration in prokaryotes, eukaryotes, or both. Typical vectors contain transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, i.e., shuttle vectors, and selection markers for both prokaryotic and eukaryotic systems.

Multispecific antibodies as described herein may be produced in prokaryotic or eukaryotic expression systems, such as bacteria, yeast, filamentous fungi, insect, and mammalian cells. It is not necessary that the recombinant antibodies of the invention be glycosylated or expressed in eukaryotic cells; however, expression in mammalian cells is generally preferred.

Examples of useful mammalian host cell lines are human embryonic kidney line (293 cells), baby hamster kidney cells (BHK cells), Chinese hamster ovary cells/−or + DHFR (CHO, CHO-S, CHO-DG44, Flp-in CHO cells), African green monkey kidney cells (VERO cells), and human liver cells (Hep G2 cells).

Mammalian tissue cell culture is preferred to express and produce the polypeptides because a number of suitable host cell lines capable of secreting intact immunoglobulins have been developed in the art, and include the CHO cell lines, various Cos cell lines, HeLa cells, preferably myeloma cell lines, or transformed B-cells or hybridomas.

In a most preferred embodiment, the multispecific, preferably bispecific, antibodies of the invention are produced by using a CHO cell line, most advantageously a CHO-S cell line.

Expression vectors for these cells can include expression control sequences, such as an origin of replication, a promoter, and an enhancer, and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like.

The vectors containing the polynucleotide sequences of interest (e.g., the heavy and light chain encoding sequences and expression control sequences) can be transferred into the host cell by well-known methods, which vary depending on the type of cellular host. For example calcium phosphate treatment or electroporation may be used for other cellular hosts. (See generally Sambrook et al., Molecular Cloning: A Laboratory Manual (Cold Spring Harbor Press, 2nd ed., 1989). When heavy and light chains are cloned on separate expression vectors, the vectors are co-transfected to obtain expression and assembly of intact immunoglobulins.

Host cells are transformed or transfected with the vectors (for example, by chemical transfection or electroporation methods) and cultured in conventional nutrient media (or modified as appropriate) for inducing promoters, selecting transformants, or amplifying the genes encoding the desired sequences.

Once expressed, the whole antibodies, their dimers, individual light and heavy chains, or other immunoglobulin forms of the present invention can be further isolated or purified to obtain preparations that are substantially homogeneous for further assays and applications. Standard protein purification methods known in the art can be used. For example, suitable purification procedures may include fractionation on immunoaffinity or ion-exchange columns, ethanol precipitation, high-performance liquid chromatography (HPLC), sodium dodecyl sulfate polyacrylamide gel electrophoresis (SDS-PAGE), ammonium sulfate precipitation, and gel filtration (see generally Scopes, Protein Purification (Springer-Verlag, N.Y., 1982). Substantially pure immunoglobulins of at least about 90 to 95% homogeneity are preferred, and 98 to 99% or more homogeneity most preferred, for pharmaceutical uses.

In vitro production allows scale-up to give large amounts of the desired multispecific, preferably bispecific, antibodies of the invention. Such methods may employ homogeneous suspension culture, e.g. in an airlift reactor or in a continuous stirrer reactor, or immobilized or entrapped cell culture, e.g. in hollow fibers, microcapsules, on agarose microbeads or ceramic cartridges.

### Therapeutic Applications

A further aspect of the invention is a pharmaceutical composition comprising a multispecific molecule, more particularly an antibody, according to the invention. Another aspect of the invention is the use of multispecific molecule, more particularly an antibody, according to the invention for the manufacture of a pharmaceutical composition. A further aspect of the invention is a method for the manufacture of a pharmaceutical composition comprising multispecific molecule, more particularly an antibody, according to the invention.

In another aspect, the present invention provides a composition, e.g. a pharmaceutical composition, containing a multispecific molecule, more particularly an antibody as defined herein, formulated together with a pharmaceutical carrier.

A composition of the present invention can be administered by a variety of methods known in the art. Any suitable route of administration is encompassed, including intravenous, oral, subcutaneous, intradermal, or mucosal administration. In another particular embodiment, an injection directly to the site of the tumor, or in its vicinity, is contemplated.

The composition of the invention is useful for treating a tumor, especially a solid tumor, such as a cancer selected from the group consisting of a lung cancer (e.g. small cell lung cancer, non-small cell lung cancer), skin cancer, melanoma, breast cancer, colorectal cancer, gastric cancer, ovarian cancer, cervical cancer, prostate cancer, kidney cancer, liver cancer, pancreatic cancer, head and neck cancer, nasopharyngeal cancer, esophageal cancer, bladder cancer, uroepithelial cancers, stomach cancer, glioma, glioblastoma, testicular, thyroid, bone, gallbladder and bile ducts, uterine, adrenal, cancers, sarcomas. Hematological malignancies (e.g. lymphoma, leukemia, multiple myeloma) are encompassed as well.

The present invention, thus generally described above, will be understood more readily by reference to the following examples, which are provided by way of illustration and are not

### EXAMPLES

Examples of multispecific constructs according to the invention are produced. The sequences of the constructs that have been produced and tested as described in the Examples below, are shown in Table 2 below.

TABLE 2

| | Heavy Chain (BiXAb) | | |
|---|---|---|---|
| Constructs | or Heavy chain without Fc (Fab-Fab) | Light Chain 1 | Light Chain 2 |
| Vα7.2/CD19 Fab-Fab | Vα7.2/CD19 Heavy | CD19 Light chain | Vα7.2 Light chain |
| | chain Fab-Fab | (SEQ ID NO: 17) | (SEQ ID NO: 15) |
| | (SEQ ID NO: 16) | | |
| Vα7.2/CD19 BiXAb | Vα7.2/CD19 Heavy | CD19 Light chain | Vα7.2 Light chain |
| | chain BiXAb | (SEQ ID NO: 17) | (SEQ ID NO: 15) |
| | (SEQ ID NO: 18) | | |
| CD19/Vα7.2 Fab-Fab | CD19/Vα7.2 Fab-Fab | CD19 Light chain | Vα7.2 Light chain |
| | Heavy chain | (SEQ ID NO: 17) | (SEQ ID NO: 15) |
| | (SEQ ID NO: 19) | | |
| CD19/Vα7.2 BiXAb | CD19/Vα7.2 BiXAb | CD19 Light chain | Vα7.2 Light chain |
| | Heavy chain | (SEQ ID NO: 17) | (SEQ ID NO: 15) |
| | (SEQ ID NO: 20) | | |
| Vα7.2/EGFR Fab-Fab | Vα7.2/EGFR Heavy | EGFR Light chain | Vα7.2 Light chain |
| | Fab-Fab chain | (SEQ ID NO: 21) | (SEQ ID NO: 15) |
| | (SEQ ID NO: 22) | | |
| EGFR/Vα7.2 Fab-Fab | EGFR/Vα7.2 Fab-Fab | EGFR Light chain | Vα7.2 Light chain |
| | Heavy chain | (SEQ ID NO: 21) | (SEQ ID NO: 15) |
| | (SEQ ID NO: 23) | | |
| Vα7.2/EGFR BiXAb | Vα7.2/EGFR Heavy | EGFR Light chain | Vα7.2 Light chain |
| | chain BiXAb chain | (SEQ ID NO: 21) | (SEQ ID NO: 15) |
| | (SEQ ID NO: 25) | | |
| EGFR/Vα7.2 BiXAb | EGFR/Vα7.2 BiXAb | EGFR Light chain | Vα7.2 Light chain |
| | Heavy chain | (SEQ ID NO: 21) | (SEQ ID NO: 15) |
| | (SEQ ID NO: 24) | | |
| Vα7.2/HER2 Fab-Fab | Vα7.2/HER2 Heavy | HER2 Light chain | Vα7.2 Light chain |
| | Fab-Fab chain | (SEQ ID NO: 26) | (SEQ ID NO: 15) |
| | (SEQ ID NO: 27) | | |
| HER2/Vα7.2 Fab-Fab | HER2/Vα7.2 Fab-Fab | HER2 Light chain | Vα7.2 Light chain |
| | Heavy chain | (SEQ ID NO: 26) | (SEQ ID NO: 15) |
| | (SEQ ID NO: 28) | | |
| Vα7.2/HER2 BiXAb | Vax7.2/Her2 Heavy | HER2 Light chain | Vα7.2 Light chain |
| | chain BiXAb chain | (SEQ ID NO: 26) | (SEQ ID NO: 15) |
| | (SEQ ID NO: 29) | | |
| HER2/Vα7.2 BiXAb | HER2/Vα7.2 BiXAb | HER2 Light chain | Vα7.2 Light chain |
| | Heavy chain | (SEQ ID NO: 26) | (SEQ ID NO: 15) |
| | (SEQ ID NO: 30) | | |

### Example 1

### Production of Anti-Vα7.2/Anti-CD19 IgG1 and Fab-Fab Gene Synthesis

The amino acid sequences of the variable regions of anti-Vα7.2 and anti-CD19 monoclonal antibodies were used to design the DNA sequences after codon optimization for mammalian expression using GeneScript program. For the heavy chain, the DNAs encoding signal peptides, variable region and constant CH1 domain of Fab1 followed the hinge linker and variable region and constant CH1 domain of Fab2 with flanking sequences for restriction enzyme digestion were synthesized by GeneScript. For the light chain, the DNAs encoding signal peptides and variable and constant Kappa regions were synthesized by GeneScript.

PCR reactions using PfuTurbo Hot Start were carried out to amplify the inserts which were then digested by NotI+ApaI and NotI+HindIII for heavy and light chains, respectively. The double digested heavy chain fragments were ligated with NotI+ApaI digested Icosagen's proprietary pQMCF expression vector in which the human IgG1 CH1+hinge+CH2+CH3 domains were already inserted for the Fc-containing molecules. For expression of Fab-Fab molecules a stop codon was inserted immediately downstream from C201 (Kabat numbering). The double digested light chain fragments were ligated with NotI+HindIII treated Icosagen's proprietary vector. Plasmid DNAs were verified by double strand DNA sequencing.

### Expression, Purification and Characterization

For a 50 mL scale expression, a total of 50 μg of plasmid DNAs in Icosagen's proprietary pQMCF vector (25 μg heavy chain+12.5 μg of each light chain, LC1 and LC2) were mixed in 1.5 mL Eppendorf tube, 1 mL of CHO TF (Xell AG) growth medium containing Icosagen's proprietary transfection Reagent 007, incubated at RT for 20 min. The mixture was loaded onto 49 mL of CHOEBNALT85 1E9 cells at 1-2×10⁶ cells/mL in 125 mL shaking flask in CHO TF (Xell AG) growth medium. Cells were shaken for 4 days at 37° C. and 6 more days at 30° C. The supernatant was harvested by centrifuging cells at 3,000 rpm for 15 min. The harvested supernatants from the Fc-containing BiXAb antibodies were purified by Protein A resin (MabSelect SuRe 5 mL column) and the supernatants from Fab-Fab antibodies were purified by CaptureSelect IgGCH1 resin. BiXAb Fc-containing antibodies we further purified employing Gel Filtration Chromatography employing Superdex 200 HiLoad 26/60 pg preparative columns, whereas the Fab-Fab antibodies where purified employing Superdex 200 Increase 10/300 GL; all antibodies were buffer-exchanged into PBS pH 7.4. All samples were sterile filtered employing 0.2 pm ULTRA Capsule GF. Electrophoresis was performed under reducing and non-reducing conditions employing 10% SDS-PAGE. Samples were prepared by combining the purified antibodies with 2× SDS sample buffer and heating for 5 min at 95° C. Preparation of reduced samples included the addition of DTT to the final concentration of 100 mM prior to heating. The apparent MW was determined using Ladder Precision Plus Protein Unstained Standards (Biorad).
Vα7.2 3C10-ML1-Light Chain is shown as SEQ ID NO: 15.
Vα7.2 3C10-ML1-AP-CD19-CC1-Heavy Chain is shown as SEQ ID N: 16.
CD19 light chain is shown as SEQ ID NO:17.
And Vα7.2 3C10-ML1-AP-CD19-CC1-Heavy chain is shown as SEQ ID NO:18.

### Example 2

### Binding of Anti-Vα7.2/Anti-CD19 Fab-Fab on CD19⁺ Cells and on Vα7.2⁺ T Cells

The anti-Vα7.2/anti-CD19 Fab-Fab produced in Example 1 was first tested for binding on CD19⁺ Raji cells. The assay was performed by flow cytometry. Briefly, Raji cells were washed with PBS and stained with a Fixable Viability Dye (eFluor™ 780, ThermoFisher) and the human Fc Block reagent (BD) (in PBS for 25 min at 4° C.). The cells were then washed with FACS buffer (PBS, 2 mM EDTA, 0.5% BSA) and stained with different concentrations of anti-Vα7.2/anti-CD19 Fab-Fab (Table 1, shown in both nM and μg/ml) for 1 h at 4° C. An irrelevant Fab-Fab was used as a negative control. Following washing (5×), the Raji cells were stained with a secondary goat anti-human antibody conjugated with Phycoerythrin (Jackson Immunoresearch) for 45 min at 4° C. The cells were then analyzed in the MACSquant flow cytometer (Miltenyi Biotec).

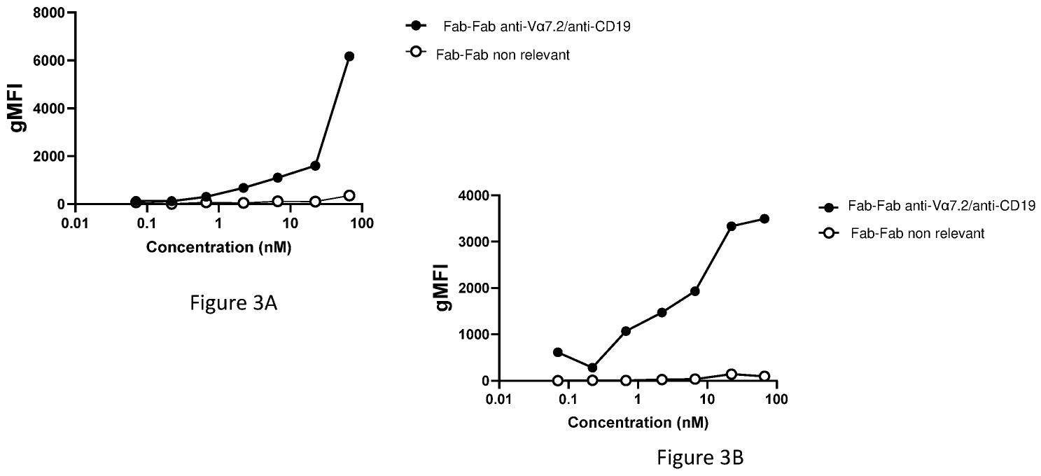

Results show a dose dependent binding of anti-Vα7.2/anti-CD19 Fab-Fab on CD19⁺ Raji cells while the irrelevant Fab-Fab did not show any binding. FIG. 3A shows a binding profile of anti-Vα7.2/anti-CD19 Fab-Fab on CD19⁺ Raji cells, expressed as normalized geometric mean fluorescence intensity, representative of four independent experiments.

The anti-Vα7.2/anti-CD19 Fab-Fab was then tested for binding on Vα7.2+CD8⁺ T cells. The assay was performed by flow cytometry. Human CD8⁺ T cells were isolated from purified peripheral blood mononuclear cells (PBMCs). Briefly, leukapheresis packs coming from healthy donors were centrifuged in ficoll gradients and the PBMCs were collected. CD8⁺ T cells were then isolated with a commercial negative selection kit (Miltenyi Biotec). These cells were then used to determine binding of anti-Vα7.2/anti-CD19 peripheral blood mononuclear cells on Vα7.2⁺ cells. Cells were washed with PBS and stained with a Fixable Viability Dye (eFluor™ 780, ThermoFisher) and the human Fc Block reagent (BD) (in PBS for 25 min at 4° C.). The cells were then washed with FACS buffer (PBS, 2 mM EDTA, 0.5% BSA) and stained with different concentrations of anti-Vα7.2/anti-CD19 Fab-Fab (Table 3, shown in both nM and μg/ml) for 1 h at 4° C. An irrelevant Fab-Fab was used as a negative control. Following washing (5×), the cells were stained with a secondary goat anti-human antibody conjugated with Phycoerythrin (Jackson Immunoresearch) and an anti-CD8 antibody (Biolegend) for 45 min at 4° C. The cells were then analyzed in the MACSquant flow cytometer.

Depending on the donor, Vα7.2⁺ cells range between 1 to 10% of CD8⁺ T cells. Results showed that anti-Vα7.2/anti-CD19 Fab-Fab could specifically detect the Vα7.2⁺ population while an irrelevant Fab-Fab could not. FIG. 3B shows a binding profile of anti-Vα7.2/anti-CD19 Fab-Fab on Vα7.2+ cells, expressed as normalized geometric mean fluorescence intensity, representative of three different donors. The EC50 calculated from two experiments was 3.49±0.2 nM.

In conclusion, anti-Vα7.2/anti-CD19 Fab-Fab can specifically bind to both of its molecular targets (CD19 and Vα7.2) expressed on the surface of live cells.

TABLE 3
Range of concentrations (in nM and μg/ml) used in
this study for anti-Vα7.2/anti-CD19 Fab-Fab

| Concentration | Concentration |
|---|---|
| (nM) | (μg/ml) |
| 0 | 0 |
| 0.07 | 0.007 |
| 0.22 | 0.021 |
| 0.67 | 0.064 |
| 2.22 | 0.213 |
| 6.67 | 0.641 |
| 22.22 | 2.137 |
| 66.67 | 6.411 |

### Example 3

### Specific MAIT Cell Activation but Minimal Overall CD8⁺ T Cell Activation by Anti-Vα7.2/Anti-CD19 Fab-Fab

Efficacy of anti-Vα7.2/anti-CD19 Fab-Fab for mediating activation of CD8⁺ T cells and specifically MAIT cells (which are CD8⁺TCRγδ⁻CD161^{hi}L18RA⁺) was evaluated in vitro. Briefly, anti-Vα7.2/anti-CD19 Fab-Fab and two antibodies, anti-Vα7.2 and anti-CD3 were coated on flat-bottomed 96-well plates at the molar concentrations (in PBS overnight at 4° C.) shown in Table 3. The anti-Vα7.2 antibody is the 3C10 clone (described in international patent application WO2008/087219), from which the anti-Vα7.2 sequence of anti-Vα7.2/anti-CD19 Fab-Fab is derived. The anti-CD3 antibody was OKT3 clone, an antibody commonly used in T cell activation assays (Saitakis et al, 2017). Before adding cells, the wells were washed at least twice with PBS.

CD8⁺ T cells were isolated as in Example 2 and added on the flat-bottomed 96-well plates (100,000 cells per well in 100 μl of RPMI 1640, 10% FBS). The wells were coated with different molar concentrations of either anti-Vα7.2/anti-CD19 Fab-Fab, or anti-CD3 or anti-Vα7.2 antibodies (Table 3). The plates were placed in the incubator at 37° C. and 5% CO₂ for 16 h. Following the culture, the cells were harvested, washed with PBS and stained first with a Fixable Viability Dye (eFluor™ 780, ThermoFisher) and the human Fc Block reagent (BD) (in PBS for 25 min at 4° C.), and then with the following antibodies (in FACS buffer at 1/100 dilution for 45 min at 4° C.): anti-CD8-PerCP-Cy5.5, anti-TCRO-FITC, anti-CD161-PE, anti-IL18RA-APC, anti-CD25-PE-Cy5 and anti-CD69-APC-Cy7 (Biolegend). The cells were then washed and analyzed in the MACSquant flow cytometer (Miltenyi Biotec).

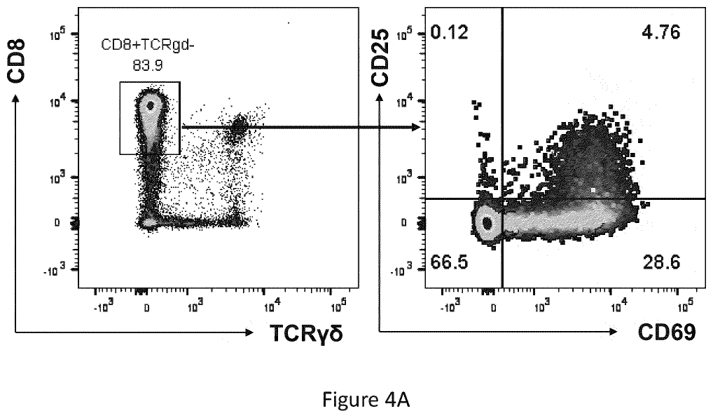

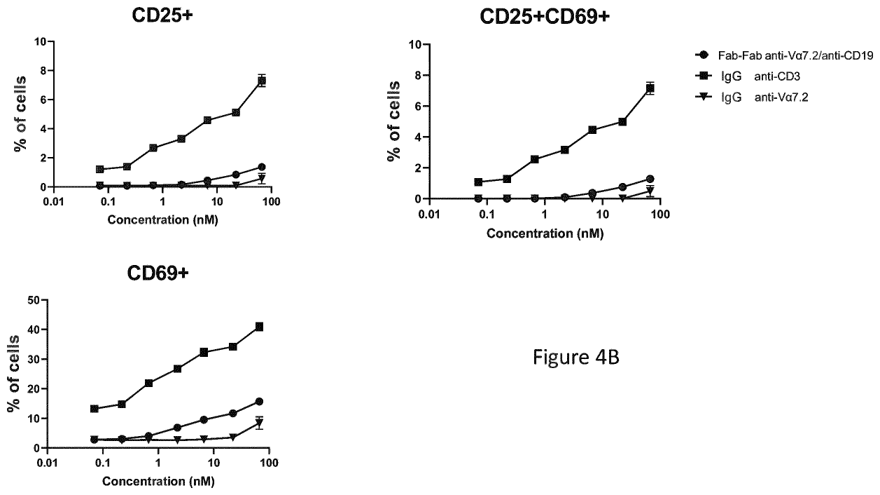

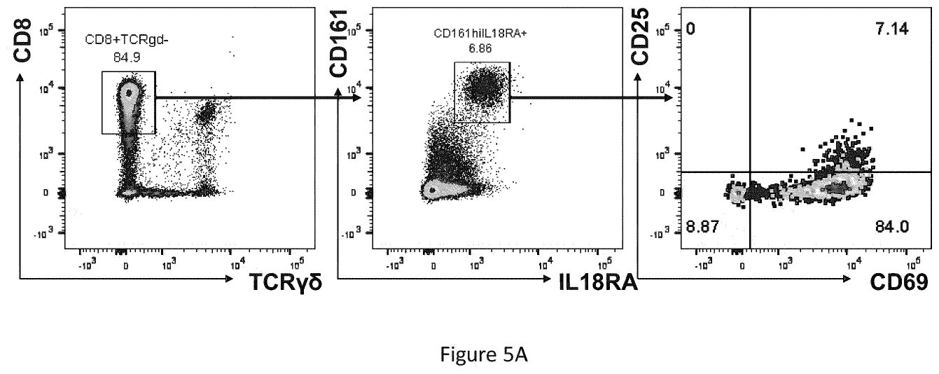

The upregulation of CD25 and CD69 is a measure of T cell activation. Therefore, following activation we looked into the percentage of cells that expressed CD25, CD69 or both. FIG. 4A shows the flow cytometry gating strategy for determining overall CD8+ T cell activation. FIG. 4B shows the percentage of CD25+, CD69⁺ and double positive (CD25+CD69+) CD8⁺TCRγδ⁻ T cells in the wells coated with different molar concentrations of either anti-Vα7.2/anti-CD19 Fab-Fab, or anti-CD3 or anti-Vα7.2 antibodies, representative of two donors. The anti-CD3 antibody was the most efficient in increasing a fraction of CD25⁺, CD69⁺ and double positive T cells, while anti-Vα7.2/anti-CD19 Fab-Fab showed, at best, four to five times less activation of total CD8⁺TCRγ∂⁻ T cells.

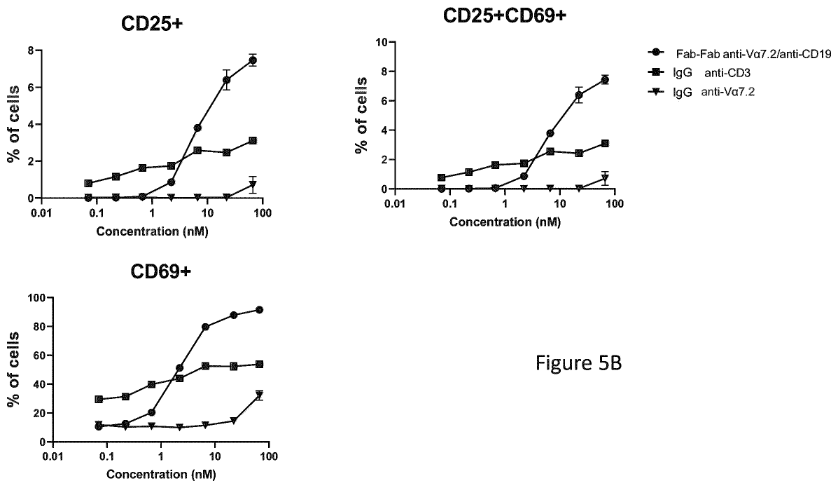

FIG. 5A shows the flow cytometry gating strategy for determining MAIT cell activation. FIG. 5B shows the percentage of CD25⁺, CD69⁺ and double positive (CD25⁺CD69⁺) MAIT (CD8⁺TCRγδ⁻CD161^{hi}IL18RA⁺) cells in the wells coated with different molar concentrations of either anti-Vα7.2/anti-CD19 Fab-Fab, or anti-CD3 or anti-Vα7.2 antibodies, representative of two donors. Anti-Vα7.2/anti-CD19 Fab-Fab was more efficient in increasing the fraction of CD25⁺, CD69⁺ and double positive MAIT cells than both monospecific antibodies.

In conclusion, anti-Vα7.2/anti-CD19 Fab-Fab can specifically activate MAIT cells and minimally activate total CD8⁺ T cells.

### Example 4

### Cytotoxicity Mediated by Anti-Vα7.2/Anti-CD19 Fab-Fab

A cytotoxic assay was set up in order to evaluate the cytotoxic potential of redirecting MAIT cells with anti-Vα7.2/anti-CD19 Fab-Fab. Briefly, human CD8⁺ T cells were isolated from purified PBMCs as described in Example 2. These cells were used in co-cultures with CD19⁺ Raji cells, engineered to express luciferase. 50,000 Raji cells were first added in U-bottomed 96-well plates in 50 μl of RPMI 1640 10% FBS. Different numbers of T cells (in 100 μl of RPMI 1640 10% FBS) were then added, corresponding to different effector:target cell ratios (Table 4). Finally, 50 μl of RPMI 1640 10% FBS containing different concentrations of anti-Vα7.2/anti-CD19 Fab-Fab (final molar concentrations as in Table 3) were added and the co-cultures were incubated at 37° C. with 5% CO₂ for 48 h. The wells were mixed with a multi-pipette and 100 ρl were transferred to a white polystyrene 96-well plate. 50 μl of PBS with luciferine (Pierce) at a final concentration of 0.1 mg/ml were added to each well and bioluminescence was measured in a SpectraMax ID3 plate reader (BioTek).

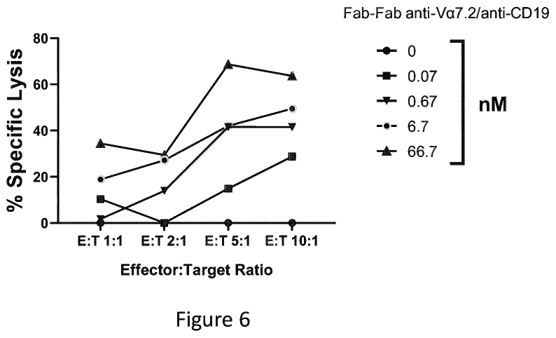

In a donor with 9% of MAIT cells among CD8⁺ T cells, anti-Vα7.2/anti-CD19 Fab-Fab promoted specific cytotoxicity with increasing dose and with increasing effector:target ratio. FIG. 6 shows the percent specific lysis after culturing Raji cells for 48 h at different concentrations of anti-Vα7.2/anti-CD19 Fab-Fab and different effector:target ratios.

In conclusion, anti-Vα7.2/anti-CD19 Fab-Fab can promote in vitro cytotoxicity of MAIT cells against CD19⁺ Raji cells.

TABLE 4
Numbers of T cells in co-cultures with
50,000 Raji cells and corresponding
effector:target cell ratios.

| Number of T cells per well | Effector: Target Ratio |
|---|---|
| 50000 | 1:1 |
| 100000 | 2:1 |
| 250000 | 5:1 |
| 500000 | 10:1 |

### Example 5

### Binding of Anti-CD19/Anti-Vα7.2-Based Bispecific Antibodies to CD19 on Tumor Cells or Vα7.2 TCR Chain on T Cells

The ability of the anti-CD19/anti-Vα7.2-based bispecific antibodies, namely anti-CD19/anti-Vα7.2 Fab-Fab, anti-Vα7.2/anti-CD19 Fab-Fab, anti-CD19/anti-Vα7.2 BiXAb and anti-Vα7.2/anti-CD19 BiXAb, to bind to the CD19 proteins expressed on the cell surface of NALM-6 tumor cells was measured using flow cytometry. Briefly, tumor cells were harvested and washed with RPMI 1640 (Gibco), 10% FBS (Eurobio), 0.1% Penicillin/Streptomycin (P/S) (Gibco). The cells were then washed with FACS buffer (PBS, 2 mM EDTA, 0.5% BSA), seeded and incubated with serial dilutions of the anti-CD19/anti-Vα7.2-based or negative control bispecific antibodies (concentrations ranging from 0 to 66 nM) at 4° C. for 45 minutes. The cells were washed and incubated with Phycoerythrin-conjugated secondary antibody (Jackson ImmunoResearch) at 4° C. for 1 hour to detect bound bispecific antibodies. A Phycoerythrin-conjugated anti-human Fc (Jackson ImmunoResearch, 109-116-098) secondary antibody was used for the detection of bound BiXAb molecules, and a Phycoerythrin-conjugated anti-human Fab (Jackson ImmunoResearch,109-116-097) antibody was used for the detection of bound Fab-Fab molecules. Cells were washed and resuspended in FACS buffer containing DAPI (Sigma), and analyzed using MACSquant flow cytometer (Miltenyi Biotec).

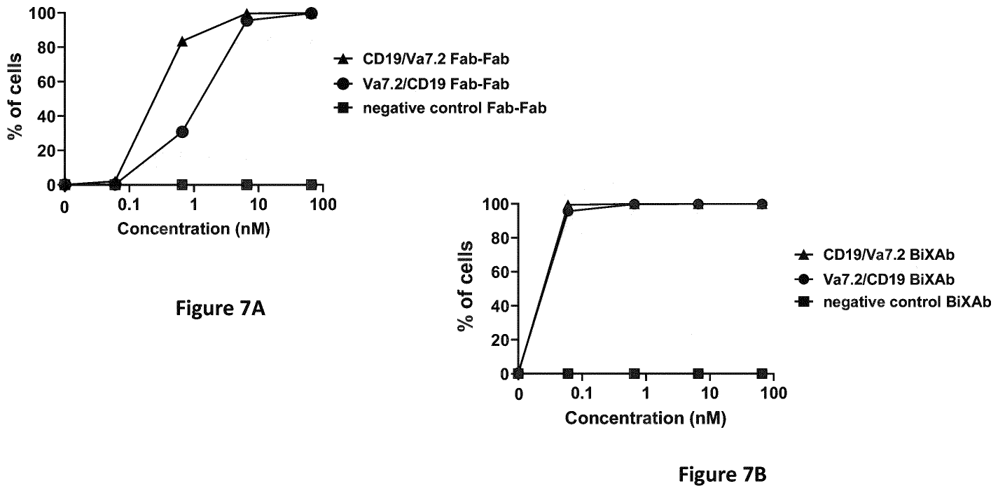

Results of the binding assay are presented in FIG. 7A and 7B for Fab-Fab and BiXAb molecules, respectively. The data are expressed as percentage of positive cells. The results demonstrated that the anti-CD19/anti-Vα7.2-based Fab-Fab and BiXAb bispecific antibodies bind to CD19 expressed on the NALM-6 cells in a dose-dependent manner. No binding was observed with the negative control Fab-Fab or BiXAb antibodies.

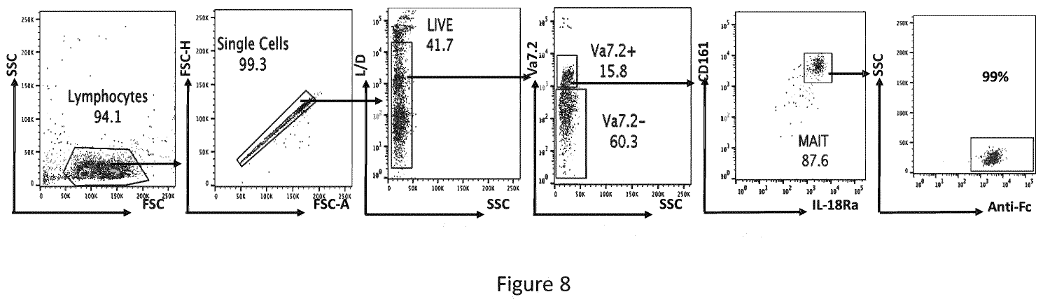

The anti-CD19/anti-Vα7.2-based BiXAb antibodies—namely anti-CD19/anti-Vα7.2 BiXAb and anti-Vα7.2/anti-CD19 BiXAb—were then tested for binding to the Vα7.2 TCR chain expressed on Vα7.2+CD8⁺ MAIT cells. The binding was determined using flow cytometry. Human CD8⁺ T cells were isolated from purified peripheral blood mononuclear cells (PBMCs). Briefly, leukapheresis packs obtained from healthy donors were centrifuged in ficoll gradients, and the PBMCs were collected. CD8⁺ T cells were isolated from PBMCs using a positive selection kit (REAlease CD8 microbead kit, Human, Miltenyi Biotec,130-117-036) according to the manufacturer's instructions. These cells were then used to assess the binding of the different BiXAb antibodies on Vα7.2⁺CD8⁺ MAIT cells. To this end, the cells were washed with FACS buffer (PBS, 2 mM EDTA, 0.5% BSA) and incubated with serial dilutions of BiXAb or negative control bispecific antibodies (concentrations ranging from 0 to 66 nM) at 4° C. for 45 minutes. The cells were then washed and incubated with Phycoerythrin-conjugated secondary antibody (Jackson ImmunoResearch) at 4° C. for 1 hour to detect bound bispecific antibodies. The cells were washed, incubated in FACS buffer containing mouse sera at room temperature for 30 minutes, washed again and stained using the following antibody panel for 30min at 4° C.: anti-human CD161-PE/Cy7 (Biolegend, HP-3G10), anti-human Vα7.2-APC/Cy7 (Biolegend, 3C10), anti-human IL18Ra-APC (Biolegend, H44). Then the cells were washed, stained with DAPI (Sigma) and analyzed using the MACSquant flow cytometer (Miltenyi Biotec). Binding results were obtained by gating on Vα7.2⁺ CD161⁺ IL-18RA cells, as shown in FIG. 8. No binding was observed outside of the Vα7.2⁺ cell population.

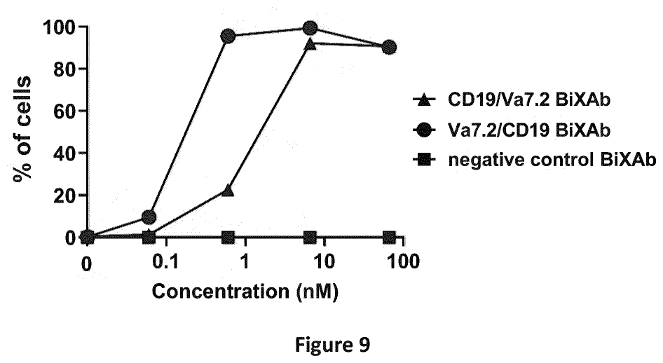

The results are represented as a percentage of positive cells and displayed in FIG. 9. The anti-CD19/anti-Vα7.2 and anti-Vα7.2/anti-CD19 BiXAbs were found to bind to the Vα7.2+CD8+ MAIT cells in dose-dependent manner. The negative control BiXAb antibody did not show any binding.

The results of the binding assays showed that anti-CD19/anti-Vα7.2-based bispecific antibodies can specifically bind to both CD19 and TCR Vα7.2 chain, expressed on the surface of live cells.

### Example 6

### MAIT Cells are Activated Following Incubation with Plate-Bound Anti-CD19/Anti-Vα7.2-Based BiXAb

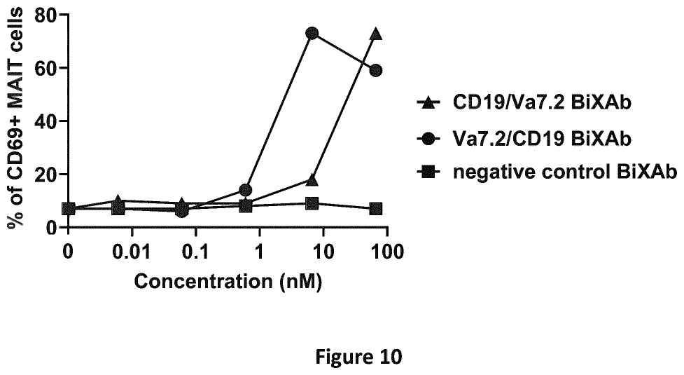

The ability of anti-CD19/anti-Vα7.2-based BiXAb antibodies—namely anti-CD19/anti-Vα7.2 BiXAb and anti-Vα7.2/anti-CD19 BiXAb—to induce MAIT cells activation was assessed by evaluating the surface expression of the activation marker CD69 after in vitro stimulation with plate-bound BiXAb antibody. Briefly, the anti-CD19/anti-Vα7.2-based BiXAbs were coated on flat-bottomed 96-well plates (in PBS, 2h at 37° C.) at concentrations ranging from 0 to 66nM. Before adding the cells, the plates were washed (×4) with PBS to remove unbound antibodies. CD8⁺ T cells were isolated form healthy donor PBMCs as in Example 5 and added on the pre-coated flat-bottomed 96-well plates (100,000 cells per well in 100 μl of RPMI 1640 (Gibco), 10% FBS (EUROBIO), 0.1% P/S (Gibco). After an 16-hour incubation at 37° C. and 5% CO2, the cells were harvested, washed with FACS buffer, and stained with a Fixable Viability Dye (Aqua, eBioscience, 65-0866-14) and the following antibody panel for 30 min at 4° C.: anti-CD3-BUV395 (BDBiosciences, UCHT1), anti-CD4-BUV737 (BDBiosciences, SK3), anti-CD8-PerCP-Cy5.5 (Biolegend, SK1), anti-TCRγδ-FITC (Biolegend, B1), anti-CD161-PE(Biolegend, HP-3G10), anti-IL18RA-APC (Biolegend, H44), anti-CD25-BV421 (Biolegend, BC96) and anti-CD69-PE/Cy7 (BDBiosciences, L78). The cells were then washed and analyzed using a Cytoflex flow cytometer (Beckman Coulter) for the expression of the activation marker CD69. As expected, the activation of T cells in this assay setting resulted in downregulation of the TCR from the cell surface. Consequently, MAIT cells were identified as CD3⁺ CD8⁺ CD161^{hi} Vα7.2⁺ cells. The activation profile of this subset is presented in FIG. 10. The results are represented as percentages of CD69+ MAIT cells. The negative control antibody did not induce the upregulation of CD69 on MAIT cells. In contrast, anti-CD19/anti-Vα7.2-based BiXAb bispecific antibodies induced a dose-dependent increase expression of CD69 on MAIT cells as shown in FIG. 10. In conclusion, plate bound anti-CD19/anti-Vα7.2 BiXAb and anti-Vα7.2/anti-CD19 BiXAb antibodies activated MAIT cells through the engagement of the anti-Vα7.2 arms of the bispecific antibody with the Vα7.2 TCR chain on MAIT cells.

### Example 7

### Redirected MAIT Cell Cytotoxicity of CD19+ Tumor Cells Upon Cross-Linking of Anti-CD19/Anti-Vα7.2-Based Bispecific Antibodies to Vα7.2 TCR Chain on MAIT Cells and CD19 on Tumor Cells

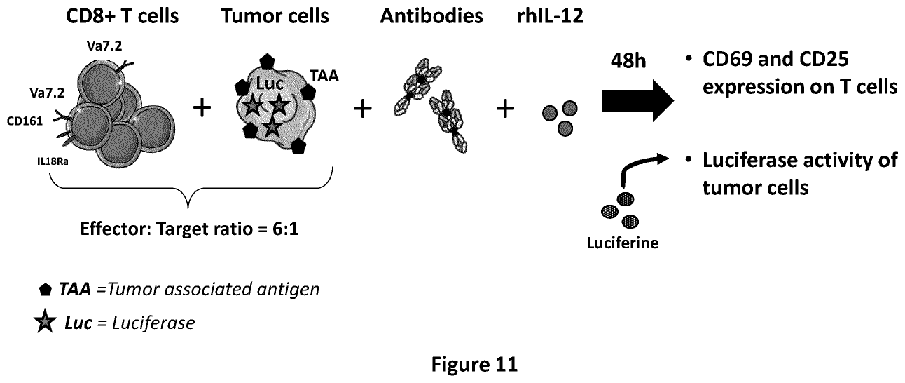

Anti-CD19/anti-Vα7.2-based bispecific antibodies, namely anti-CD19/anti-Vα7.2 Fab-Fab, anti-Vα7.2/anti-CD19 Fab-Fab, anti-CD19/anti-Vα7.2 BiXAb and anti-Vα7.2/anti-CD19 BiXAb were analyzed for their ability to induce MAIT cell-mediated apoptosis in CD19-expressing tumor cells upon crosslinking of the construct via binding of anti-CD19 moieties to CD19 on A-549 tumor cells. In addition, the ability of the bispecific antibodies to induce MAIT cell activation was assessed by evaluating the surface expression of the activation markers CD69 and CD25. Briefly, human CD8⁺ T cells were isolated from purified PBMCs, as described in Example 5. These cells were co-cultured with A-549 tumor cells engineered to express CD19 and luciferase. 105A-549 tumor cells were first added in white polystyrene 96-well plate in 50 μl of RPMI 1640 (Gibco), 10% FBS (EUROBIO) 0.1% P/S (Gibco). 6×10⁵ CD8⁺ T cells in 100 μl of RPMI 1640 (Gibco), 10% FBS (EUROBIO), 0.1% P/S (Gibco), recombinant human interleukin 12 (rhIL-12) 30 ng/mL (Peprotech) were then added, corresponding to an effector:target cell ratio of 6:1. Finally, 50 μl of RPMI 1640 (Gibco), 10% FBS (Eurobio), 0.1% P/S (Gibco), IL-12 30 ng/mL (Peprotech) containing different concentrations of bispecific antibodies (final molar concentrations ranging from 0 to 66 nM) were added. The plates were incubated at 37° C. with 5% CO2 for 48h. Supernatants were discarded, and cells were washed in PBS. Then the cells were resuspended in 50 μl of RPMI 1640 (Gibco), 10% FBS (Eurobio), 0.1% P/S (Gibco) in white polystyrene 96-well plate. 50 μl of PBS containing luciferine (Perkin elmer) at a final concentration of 0.1 mg/ml were added to each well, and bioluminescence was measured in a SpectraMax 1D3 plate reader (BioTek). An overview of the experimental setup is presented on FIG. 11. The CD8+ T cell and tumor cell co-culture was also analyzed using flow cytometry. For that purpose, the cells were harvested, washed with FACS buffer, and stained with a Fixable Viability Dye (Aqua, eBioscience, 65-0866-14) and the following antibody panel for 30 min at 4° C.: anti-CD3-BUV395 (BDBiosciences UCHT1), anti-CD4-BUV737 (BDBiosciences, SK3), anti-CD8-PerCP-Cy5.5 (Biolegend, SK1), anti-TCRγδ-FITC (Biolegend, B1), anti-CD161-PE (Biolegend, HP-3G10), anti-IL18RA-APC (Biolegend, H44), anti-CD25-BV421 (Biolegend, BC96) and anti-CD69-PE/Cy7 (BDBiosciences, L78). The cells were then washed and analyzed by flow cytometry (Cytoflex, Beckman Coulter) to measure the expression of the activation markers CD69 and CD25 on MAIT cells.

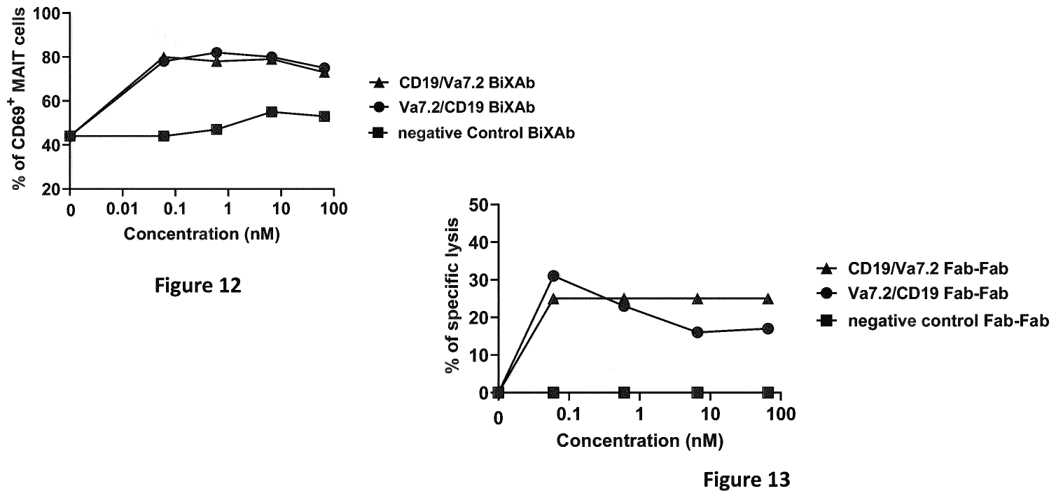

The activation of MAIT cells following the co-culture was analyzed as described in Example 6. The results for the BiXAb antibodies are reported in FIG. 12, respectively. The results are represented as percentages of single-positive CD69 MAIT cells. The addition of the negative control BiXAb antibodies to the co-culture did not activate MAIT cells, as shown by the absence of upregulation of CD69 on MAIT cells. As shown in FIG. 12, the addition of the anti-CD19/anti-Vα7.2-based BiXAb promoted MAIT cell activation at the tested concentrations. Similarly, the anti-CD19/anti-Vα7.2-based Fab-Fab induced an upregulation of the activation markers CD69 and CD25 on MAIT cells.

In addition, the percentage of CD19+ A-549 tumor cells lysis was assessed by adding luciferin to the culture and measuring the level of luciferase activity in living tumor cells into the co-culture well. The percentage of lysis is reported in FIG. 13 for the Fab-Fab bispecific antibodies. Percentages of up to 30% lysis were reached at concentration of the anti-CD19/anti-Vα7.2 Fab-Fab, anti-Vα7.2/anti-CD19 Fab-Fab as low as 0.06 nM. Similarly, the addition of the anti-CD19/anti-Vα7.2 BiXAb and anti-Vα7.2/anti-CD19 BiXAb to the co-culture induce a maximal tumor lysis of up to 30% at concentrations as low as 0.06 nM.

Altogether, these results show that the anti-CD19-based bispecific antibodies direct MAIT cell cytotoxicity towards CD19-expressing tumor cells.

### Example 8

### Production of IgG1 (BiXAb) and Fab-Fab Targeting Vα7.2, EGFR or HER2

The amino acid sequences of the variable regions of anti-Vα7.2, anti-EGFR and anti-HER2 monoclonal antibodies were used to design the following IgG1 BiXAb and Fab-Fab bispecific antibodies:
Anti-Vα7.2/anti-EGFR Fab-Fab
Anti-EGFR/anti-Vα7.2 Fab-Fab
Anti-Vα7.2/anti-EGFR BiXAb
Anti-EGFR/anti-Vα7.2 BiXAb
Anti-Vα7.2/anti-HER2 Fab-Fab
Anti-HER2/anti-Vα7.2 Fab-Fab
Anti-Vα7.2/anti-HER2 BiXAb
Anti-HER2/anti-Vα7.2 BiXAb

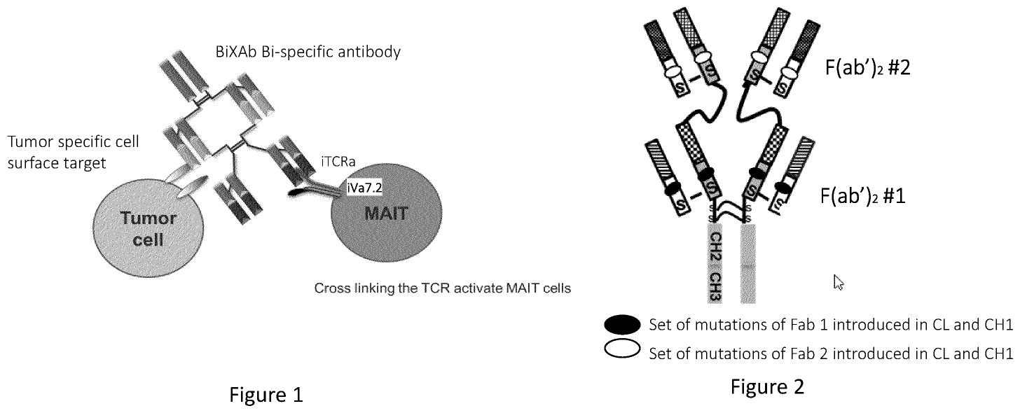

The names reflect the position of each binding moiety: For instance, anti-Vα7.2/anti-TAA Fab-Fab or BixAb means that the anti-Vα7.2 binding fragment is positioned at the N-terminus (see FIG. 1). Conversely, anti-TAA/anti-Vα7.2 Fab-Fab or BixAb means that the anti-TAA binding fragment is positioned at the N-terminus.

See Table 2 for a reference to the sequences.

In addition, the negative control BiXAb and Fab-Fab antibodies were generated using the sequence of the variable region of the humanized monoclonal antibody anti-RSV, MEDI-493. All the BiXAb comprised a LALA mutation in the CH2 domain. The introduction of the LALA mutation in the CH2 domain of human IgG1 is known to reduce Fcγ receptor binding (Bruhns, et al., 2009 and Hezareh et al., 2001).

The methods used to perform the gene synthesis, expression, purification and characterization of these bispecific antibodies were as described in Example 1.

### Example 9

### Binding of the Anti-HER2/Anti-Vα7.2-Based Bispecific Antibodies to HER2 on Tumor Cells or Vα7.2 TCR Chain on T Cells

The ability of the anti-HER2/anti-Vα7.2-based bispecific antibodies, namely anti-HER2/anti-Vα7.2 Fab-Fab, anti-HER2/anti-Vα7.2 BiXAb and anti-Vα7.2/anti-HER2 BiXAb, to bind to the HER2 protein expressed on the cell surface of A-549 tumor cells and the Vα7.2 TCR chain expressed on Vα7.2+CD8+ MAIT cells was measured using flow cytometry. The experiments were performed as described in Example 5.

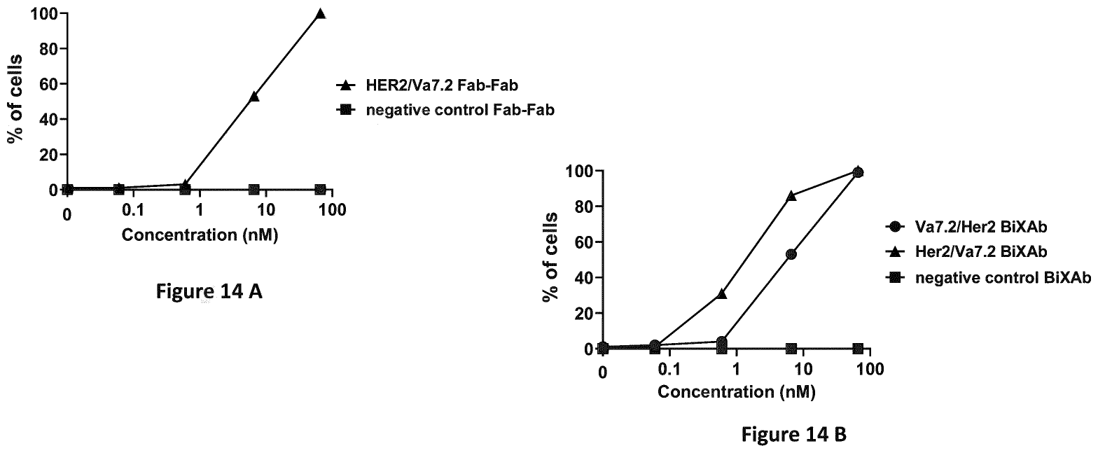

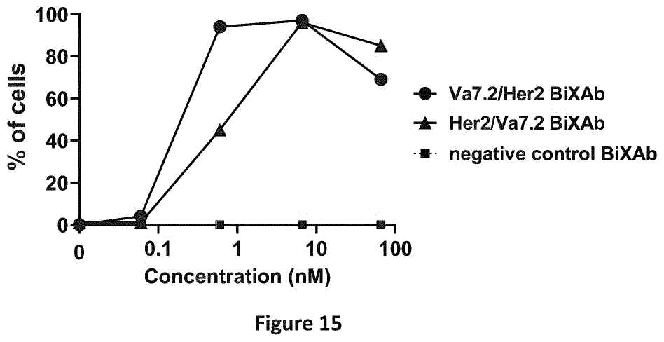

The results of the binding of anti-HER2/anti-Vα7.2-based bispecific molecules to HER2-expressing tumor cells are shown in FIGS. 14A and 14B, for Fab-Fab and BiXAb molecules, respectively. The results are represented as a percentage of positive cells. While the negative control Fab-Fab or BiXAb antibodies did not show any binding, all the anti-HER2/anti-Vα7.2-based bispecific antibodies show a dose dependent binding on HER2+ A-549 cells. Additionally, as shown in FIG. 15, both anti-HER2/anti-Vα7.2-based BiXAb bispecific antibodies demonstrated a dose dependent binding to the Vα7.2+CD8+ MAIT cells through the anti-Vα7.2 arm of the antibodies. The negative control BiXAb antibody did not show any cell binding. The results are represented as a percentage of positive cells.

In summary, the results of the binding assays demonstrated that anti-HER2/anti-Vα7.2-based bispecific antibodies can specifically bind to both HER2 and TCR Vα7.2 chain, expressed on the surface of HER2 expressing tumor cells and MAIT cells, respectively.

### Example 10

### MAIT Cells Are Activated Following Incubation With Plate-Bound Anti-HER2/Anti-Vα7.2-Based BiXAb

The ability of anti-HER2/anti-Vα7.2-based BiXAbs, namely anti-HER2/anti-Vα7.2 BiXAb and anti-Vα7.2/anti-HER2 BiXAb, to activate MAIT cells was evaluated in vitro with plate-bound BiXAb antibodies, as described in Example 6.

The stimulation of the MAIT cells with the anti-HER2/anti-Vα7.2-based BiXAbs induced dose-dependent upregulation of the activation markers CD69 and CD25 demonstrating that plate-bound anti-HER2/anti-Vα7.2 BiXAb and anti-Vα7.2/anti-HER2 BiXAb antibodies ex vivo activated MAIT cells through the engagement of the anti-Vα7.2 arms of the bispecific antibodies with the Vα7.2 TCR chain on MAIT cells.

### Example 11

### Redirected MAIT Cell Cytotoxicity of HER2+ Tumor Cells Upon Cross-Linking of Anti-HER2/Anti-Vα7.2-Based Bispecific Antibodies to Both Vα7.2 on MAIT Cells and HER2 on Tumor Cells

Following the same protocol described in Example 7, a cytotoxic assay was performed to evaluate the potential of the different anti-HER2/anti-Vα7.2-based bispecific antibodies, namely anti-HER2/anti-Vα7.2 Fab-Fab, anti-Vα7.2/anti-HER2 Fab-Fab, anti-HER2/anti-Vα7.2 BiXAb and anti-Vα7.2/anti-HER2 BiXAb, to activate and redirect MAIT cell cytotoxic activity against tumor target cells. The A-549 tumor cell line engineered to express luciferase was used as a target cell line.

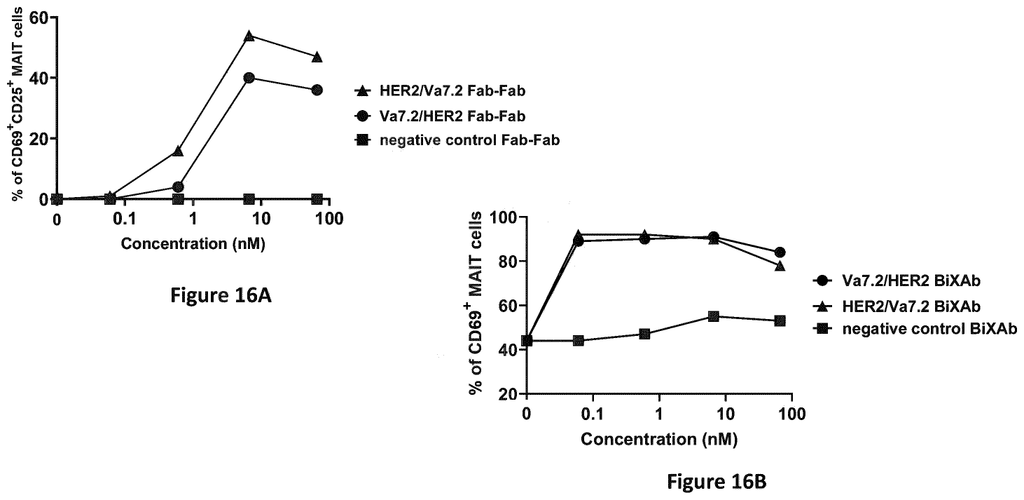

The activation of MAIT cells following the co-culture was analyzed as described above for CD8⁺ T cells. FIGS. 16A and 16B display the results for the Fab-Fab antibodies and for the BiXAb antibodies, respectively. The results are represented as percentages of double-positive CD25+CD69+ or single-positive CD69+ MAIT cells. The addition of the negative control Fab-Fab or BiXAb antibodies in the co-culture did not activate MAIT cells, as shown by the absence of upregulation of CD69 and CD25 on MAIT cells. In contrast, the addition of the anti-HER2/anti-Vα7.2-based bispecific antibodies in the co-culture promoted MAIT cell activation at the tested doses. The BiXAb molecules induced a maximal response at concentrations as low as 0.06 nM.

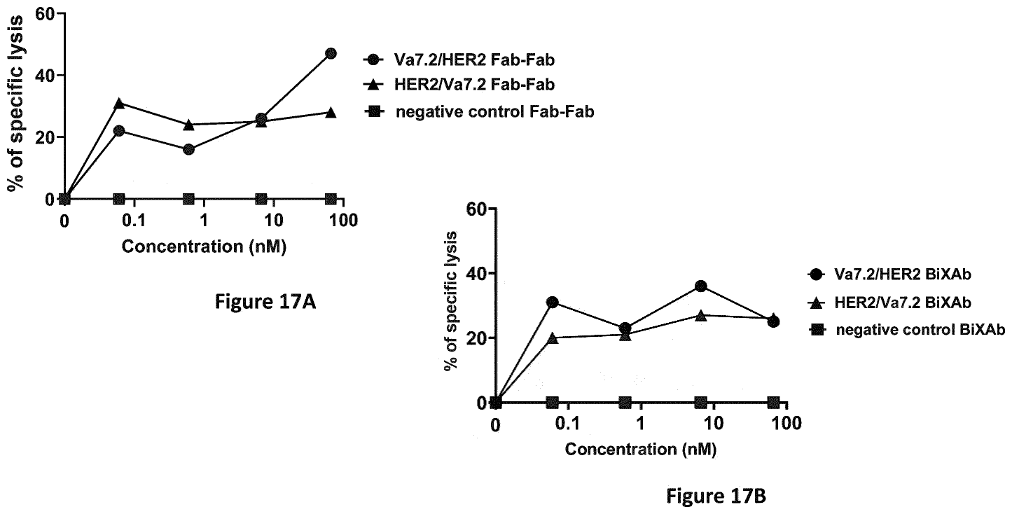

In addition, the percentage of HER2+ A-549 tumor cells lysis was assessed by adding luciferin and measuring the luciferase activity in living tumor cells in the co-culture well. The percentage of lysis is reported in FIGS. 17A and 17B for the Fab-Fab and BiXAb, respectively.

Percentages of up to 31% lysis were reached at concentration of the anti-HER2/anti-Vα7.2 Fab-Fab or BiXAb, anti-Vα7.2/anti-HER2 Fab-Fab or BiXAb as low as 0.06 nM.

Altogether, these results show that the anti-HER2/anti-Vα7.2-based bispecific antibodies redirect MAIT cell cytotoxicity towards HER2-expressing tumor cells.

### Example 12

### Binding of the Anti-EGFR/AntiVα7.2-Based Bispecific Antibodies to EGFR on Tumor Cells or Vα7.2 TCR Chain on T Cells

The ability of the anti-EGFR/anti-Vα7.2-based bispecific antibodies, namely anti-Vα7.2/anti-EGFR BiXAb and anti-EGFR/anti-Vα7.2 BiXAb, to bind to the EGFR protein expressed on the cell surface of A-549 tumor cells and the Vα7.2 TCR chain expressed on Vα7.2+CD8⁺ T cells was measured using flow cytometry. The experiments were performed as described in Example 5.

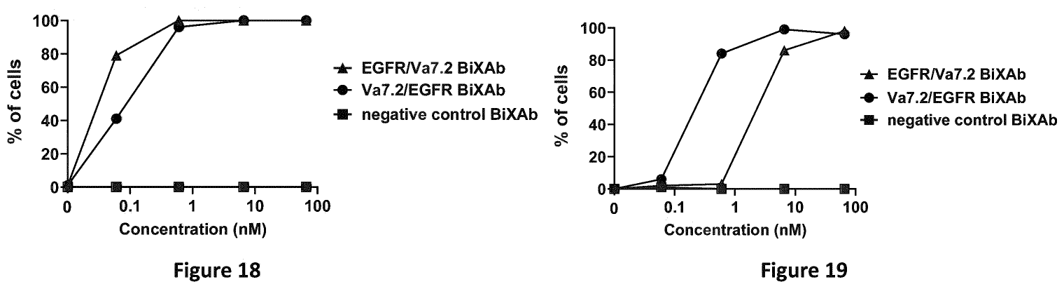

The results of the binding of anti-EGFR/anti-Vα7.2-based bispecific antibodies to EGFR-expressing tumor cells are displayed in FIG. 18. The results are represented as a percentage of positive cells. The anti-EGFR/anti-Vα7.2-based bispecific antibodies were found to bind cell-surface expressed EGFR in a dose dependent manner. The negative control BiXAb antibodies did not show any binding.

Additionally, as shown in FIG. 19, anti-EGFR/anti-Vα7.2-based BiXAb bispecific antibodies were found to bind the Vα7.2 TCR chain expressed by the CD8+ MAIT cells. The negative control BiXAb antibody did not show any binding. The results are represented as a percentage of positive cells.

The results of the binding assays showed that anti-EGFR/anti-Vα7.2-based bispecific antibodies can specifically bind to both EGFR and TCR Vα7.2 chain, expressed on the surface of live cells.

### Example 13

### Redirected MAIT Cell Cytotoxicity of EGFR+ Tumor Cells Upon Cross-Linking of Anti-EGFR/Anti-Vα7.2-Based Bispecific Antibodies to Both Vα7.2 on MAIT Cells and EGFR on Tumor Cells

Following the same protocol as described in Example 7, a cytotoxic assay was performed to evaluate the ability of the anti-EGFR/anti-Vα7.2-based bispecific antibodies, to activate and redirect MAIT cell cytotoxic activity against tumor target cells. The EGFR expressing A-549 tumor cell line engineered to express luciferase was used as a target cell line.

Adding anti-Vα7.2/anti-EGFR BiXAb to the co-culture triggered the cytolytic function of the MAIT cells by redirecting them against the tumor cells at a concentration as low as 0.6 nM. A maximal specific lysis of up to 49% was achieved at a concentration of 6 nM.

### Example 14

### MAIT Cells Displayed Cytotoxic Effects Towards Tumor Cells In Vivo

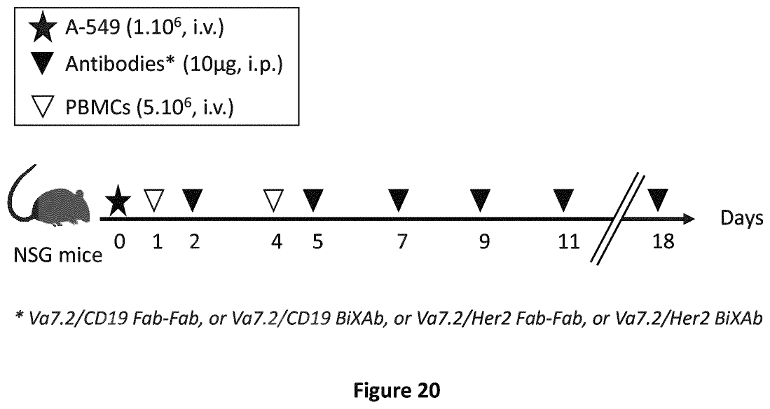

Six 8- to 12-week-old female NSG mice (nonobese diabetic severe combined immunodeficiency gamma [NOD.Cg-Prkdcscid IL2rgtm1Wjl/SzJ)) were used for each group. All mice from the same treatment group were co-housed in the same cage. For this experiment, PBMCs were obtained from a single healthy donor. After tumor implantation (1×10⁶ HER2+ A-549 tumor cells expressing CD19 and luciferase, 100 μl in PBS injected into the tail vein), mice were treated with the intravenous injection of 5×10⁶ human PBMCs in 100 μl in PBS on days 2 and 4, and with intraperitoneal injections of antibodies (10 μg of antibodies per injection in 100 μl in PBS, a total of 5 injections on days 2, 5, 7, 9, 10, and 18), as described in FIG. 20. Mice were monitored every 2-3 days for weight loss and every day for overall health. Tumor progression was followed twice a week by monitoring luciferase activity of implanted tumor cells. Briefly, mice were intraperitoneally injected with 100 μl of D-Luciferin Firefly Potassium salt in PBS (30 mg/kg, Perkin Elmer Ref. 122 799), bioluminescence images were acquired using IVIS® Lumina II In Vivo Imaging System and luciferase expression was analyzed with the Living Image® software (Perkin Elmer). During this process, mice where under general anesthesia. Mice were sacrificed when body weight loss was more than 20%. No treatment related toxicities were observed in mice throughout the experiment.

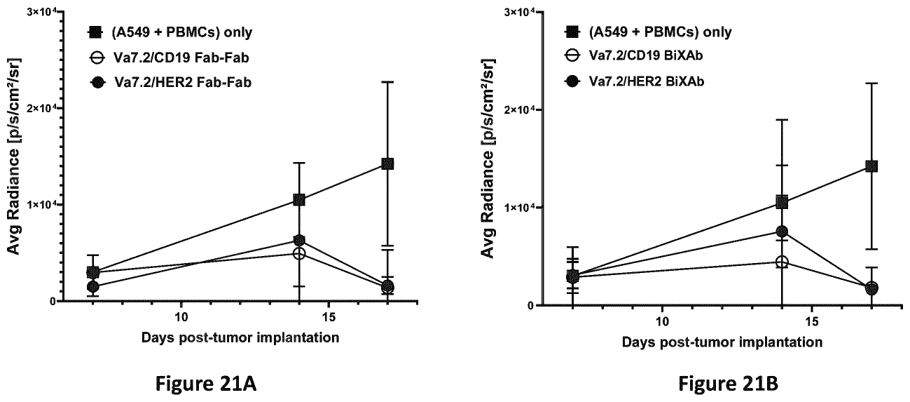

As shown in FIGS. 21A and 21B, in tumor bearing mice injected with PBMC without any antibody treatment, the bioluminescence signal increased in most mice overtime. In contrast, in animals that were treated with the anti-Vα7.2/anti-CD19 Fab-Fab, anti-Vα7.2/anti-HER2 Fab-Fab, anti-Vα7.2/anti-CD19 BiXAb, anti-Vα7.2/anti-HER2 BiXAb, the tumor showed a slower growth before regressing at day 17 post tumor implantation. At day 17, the large majority of the animals treated with the bispecific antibodies showed weak or no bioluminescence signal.

### REFERENCES

Bruhns P, Iannascoli B, England P, Mancardi D A, Fernandez N, Jorieux S, Daeron M. Specificity and affinity of human Fey receptors and their polymorphic variants for human IgG subclasses. Blood 1 13(16), 3716-25 (2009).
Choi B D, Gedeon P C, Herndon J E, Archer G E, Reap E A, Sanchez-Perez L, Mitchell D A, Bigner D D, Sampson J H: Human regulatory T cells kill tumor cells through granzyme-dependent cytotoxicity upon retargeting with a bispecific antibody. Cancer Immunol Res 2013, 1:163.
Dusseaux et al, 2011, Blood. 27;117(4):1250-9
Franciszkiewicz K, Salou M, Legoux F, Zhou Q, Cui Y, Bessoles S, Lantz O. MHC class I-related molecule, MR1, and mucosal-associated invariant T cells. Immunol Rev. 2016 July;272(1):120-38.
Koristka S, Cartellieri M, Theil A, Feldmann A, Arndt C, Stamova S, Michalk I, Topfer K, Temme A, Kretschmer K et al.: Retargeting of human regulatory T cells by single-chain bispecific antibodies. J Immunol 2012, 188:1551-1558.
Koristka S, Cartellieri M, Arndt C, Bippes C C, Feldmann A, Michalk I, Wiefel K, Stamova S, Schmitz M, Ehninger G et al.: Retargeting of regulatory T cells to surface-inducible autoantigen La/SS-B. J Autoimmun 2013, 42:105-116.
Krupka C, Kufer P, Kischel R, Zugmaier G, Bo″geholz J, Köhnke T, Lichtenegger F S, Schneider S, Metzeler K H, Fiegl M et al.: CD33 target validation and sustained depletion of AML blasts in long-term cultures by the bispecific T-cell-engaging antibody AMG 330. Blood 2014, 123:356-365.
Hezareh M, Hessell A J, Jensen R C, van de Winkel J G, Parren P W. Effector function activities of a panel of mutants of a broadly neutralizing antibody against human immunodeficiency virus type 1. J. Virol. 75(24), 12161-8 (2001)
Le Bourhis et al, PLoS Pathogens, 2013, Volume 9, Issue 10, e1003681
Michalk I, Feldmann A, Koristka S, Arndt C, Cartellieri M, Ehninger A, Ehninger G, Bachmann MvP: Characterization of a novel single-chain bispecific antibody for retargeting of T cells to tumor cells via the TCR co-receptor CD8. PLOS ONE 2014, 9:e95517.
Nagorsen D, Baeuerle P A: Immunomodulatory therapy of cancer with T cell-engaging BiTE antibody blinatumomab. Exp Cell Res 2011, 317:1255-1260.
Offner S, Hofmeister R, Romaniuk A, Kufer P, Baeuerle P A: Induction of regular cytolytic T cell synapses by bispecific single-chain antibody constructs on MHC class I-negative tumor cells. Mol Immunol 2006, 43:763-771.
Reantragoon et al, 2013, J Exp Med. 210(11):2305-20
Saitakis M, Dogniaux S, Goudot C, Bufi N, Asnacios S, Maurin M, Randriamampita C, Asnacios A and Hivroz C: Different TCR-induced T lymphocyte responses are potentiated by stiffness with variable sensitivity. Elife, 2017;6:e23190
Salou M, Franciszkiewicz K and Lantz O: MAIT cells in infectious diseases. Curr Opinion Immunol, 2017;48:7-14.
Salou et al, J Exp Med. 2019;216(1):133-151
Tilloy et al. (1999) J. Exp. Med. 189(12): 1907-1921

1-9. (canceled)

20. A multispecific molecule capable of simultaneous binding to a Mucosal Associated Invariant T (MAIT) cell and a tumor cell, said multispecific molecule comprising at least one domain that specifically binds a Vα7.2 T cell receptor (TCR) and at least one domain that specifically binds a tumor associated antigen (TAA).

21. The multispecific molecule of claim 20, which is a multispecific antibody or an antigen-binding fragment thereof.

22. The multispecific molecule of claim 20, said multispecific molecule comprising at least one multispecific antigen-binding fragment comprising at least two Fab fragments with different CH1 and CL domains, wherein said Fab fragments are tandemly arranged in any order, the C-terminal end of the CH1 domain of a first Fab fragment being linked to the N-terminal end of the VH domain of the following Fab fragment through a polypeptide linker, wherein at least one Fab fragment binds Vα7.2, and at least another Fab fragment binds the TAA.

23. The multispecific molecule of claim 22, said multispecific molecule consisting of at least one multispecific antigen-binding fragment comprising at least two Fab fragments with different CH1 and CL domains, wherein said Fab fragments are tandemly arranged in any order, the C-terminal end of the CH1 domain of a first Fab fragment being linked to the N-terminal end of the VH domain of the following Fab fragment through a polypeptide linker, wherein at least one Fab fragment binds Vα7.2, and at least another Fab fragment binds the TAA.

24. The multispecific molecule of claim 22, said multispecific molecule comprising two identical antigen-binding arms, each consisting of a multispecific antigen-binding fragment.

25. The multispecific molecule of claim 20, wherein the domain that binds a Vα7.2 TCR binds Vα7.2-Jα33, Vα7.2-Jα20 or Vα7.2-Jα12.

26. The multispecific molecule of claim 25, said multispecific molecule competing or binding to the same or substantially the same epitope of the Vα7.2-Jα33 polypeptide as monoclonal antibody 3C10.

27. The multispecific molecule of claim 25, wherein the heavy variable chain of the anti-Vα7.2 domain comprises the following CDRs: GFNIKDTH (SEQ ID NO: 4); TDPASGDT (SEQ ID NO:5); and CAHYYRDDVNYAMDY (SEQ ID NO:6); and/or the light variable chain of the anti-Vα7.2 domain comprises the following CDRs: QNVGSN (SEQ ID NO:7); SSS; and QQYNTYPYT (SEQ ID NO:8).

28. The multispecific molecule of claim 20, wherein the TAA is a tumor cell surface antigen that is expressed on hematological malignancies or solid tumor cells.

29. The multispecific molecule of claim 28, wherein the TAA is selected from the group consisting of CD19, CD20, CD38, EGFR, HER2, VEGF, CD52, CD33, RANK-L, GD2, CD33, CEA, CEACAM1, CEACAMS, PSG, MUC1, PSCA, PSMA, GPA33, CA9, PRAME, CLDN1, HER3, glypican-3, CD22, CD25, CD40, CD30, CD79b, CD138 (syndecan-1), BCMA, SLAMF7 (CS1, CD319), CD56, CCR4, EpCAM, PDGFR-a, Apo2L/TRAIL, and PD-L1.

30. The multispecific molecule of claim 29, wherein the TAA is CD19.

31. The multispecific molecule of claim 29, wherein the TAA is EGFR.

32. The multispecific molecule of claim 29, wherein the TAA is HER2.

33. A polypeptide comprising a heavy chain of the antigen-binding fragment or a heavy chain of the multispecific antibody of claim 21.

34. A polynucleotide encoding the polypeptide of claim 33.

35. A host cell transfected with an expression vector comprising the polynucleotide of claim 34.

36. A method for producing an antigen-binding fragment or a multispecific antibody, said method comprising the following steps: a) culturing in suitable medium and culture conditions a host cell expressing an antibody heavy chain and an antibody light chain of claim 21; and b) recovering said antibodies from the culture medium or from said cultured cells.

37. A method of treating a tumor comprising administering a multispecific molecule of claim 20 to a patient having a tumor.

38. The method of claim 37, wherein the tumor is a solid tumor.


SEQUENCE LISTING (30 sequences)

SEQ ID NO: 1 - protein, 110 aa, Homo sapiens
  misc_feature (91)..(92): Xaa can be any naturally occurring amino acid
QNIDQPTEMTATEGAIVQINCTYQTSGFTFNGLFWYQQHAGEAPTFLSYNVLDGLEEKGRFSSFLSRSKGYSYLLLKELQMKDSASYLCAXXDSNYQLIWGAGTKLIIKP

SEQ ID NO: 2 - protein, 109 aa, Homo sapiens
  misc_feature (91)..(92): Xaa can be any naturally occurring amino acid
QNIDQPTEMTATEGAIVQINCTYQTSGFTFNGLFWYQQHAGEAPTFLSYNVLDGLEEKGRFSSFLSRSKGYSYLLLKELQMKDSASYLCAXXDSSYKLIFGSGTRLVRP

SEQ ID NO: 3 - protein, 110 aa, Homo sapiens
  misc_feature (92)..(94): Xaa can be any naturally occurring amino acid
QNIDQPTEMTATEGAIVQINCTYQTSGFTFNGLFWYQQHAGEAPTFLSYNVLDGLEEKGRFSSFLSRSKGYSYLLLKELQMKDSASYLCAVXXXDYKLSFGAGTTVTVRA

SEQ ID NO: 4 - protein, 8 aa, Homo sapiens
GFNIKDTH

SEQ ID NO: 5 - protein, 8 aa, Homo sapiens
TDPASGDT

SEQ ID NO: 6 - protein, 15 aa, Homo sapiens
CAHYYRDDVNYAMDY

SEQ ID NO: 7 - protein, 6 aa, Homo sapiens
QNVGSN

SEQ ID NO: 8 - protein, 9 aa, Homo sapiens
QQYNTYPYT

SEQ ID NO: 9 - protein, 34 aa, Artificial Sequence
  Linker
  misc_feature (4)..(4): Xaa can be any naturally occurring amino acid
  misc_feature (8)..(8): Xaa can be any naturally occurring amino acid
  misc_feature (10)..(11): Xaa can be any naturally occurring amino acid
  misc_feature (14)..(14): Xaa can be any naturally occurring amino acid
  misc_feature (24)..(25): Xaa can be any naturally occurring amino acid
  misc_feature (28)..(28): Xaa can be any naturally occurring amino acid
  misc_feature (30)..(30): Xaa can be any naturally occurring amino acid
  misc_feature (32)..(32): Xaa can be any naturally occurring amino acid
EPKXCDKXHXXPPXPAPELLGGPXXPPXPXPXGG

SEQ ID NO: 10 - protein, 34 aa, Artificial Sequence
  linker
EPKSCDKTHTSPPAPAPELLGGPGGPPGPGPGGG

SEQ ID NO: 11 - protein, 34 aa, Artificial Sequence
  linker
EPKSCDKTHTSPPAPAPELLGGPAAPPAPAPAGG

SEQ ID NO: 12 - protein, 34 aa, Artificial Sequence
  linker
EPKSCDKTHTSPPAPAPELLGGPAAPPGPAPGGG

SEQ ID NO: 13 - protein, 34 aa, Artificial Sequence
  linker
EPKSCDKTHTCPPCPAPELLGGPSTPPTPSPSGG

SEQ ID NO: 14 - protein, 34 aa, Artificial Sequence
  linker
EPKSCDKTHTSPPSPAPELLGGPSTPPTPSPSGG

SEQ ID NO: 15 - protein, 214 aa, Artificial Sequence
  Valpha7.2 3C10-ML1-Light Chain
DIVMTQSQKFLSTSVGDRVSVTCKARQNVGSNVAWYQQKPGQSPKSLIYSSSFRYSGVPDRFTGSGSGTDFTLTISNVQSEDLAEYFCQQYNTYPYTFGGGTELEIKRTVAAPSVFIFPPSDEQLKSGTASVTCLLNNFYPREAKVQWKVDNALQSGNSQESVTEQDSKDSTYSLVSTLTLSKADYEKHKVYACEVTHQGLSSPVTKSFNRGEC

SEQ ID NO: 16 - protein, 480 aa, Artificial Sequence
  Valpha7.2/CD19 heavy chain Fab-Fab
EVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTAPPAPAPELLGGPAAPPAPAPAGGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSC

SEQ ID NO: 17 - protein, 218 aa, Artificial Sequence
  CD19 light chain
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 707 aa, Artificial Sequence
  Valpha7.2 /CD19 heavy chain BiXab
EVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTAPPAPAPELLGGPAAPPAPAPAGGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 480 aa, Artificial Sequence
  CD19/Valpha7.2 Heavy chain Fab-Fab
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSCDKTHTAPPAPAPELLGGPAAPPAPAPAGGEVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSC

SEQ ID NO: 20 - protein, 707 aa, Artificial Sequence
  CD19/Valpha7.2 BiXAb Heavy chain
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSCDKTHTAPPAPAPELLGGPAAPPAPAPAGGEVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 213 aa, Artificial Sequence
  EGFR Light chain
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 477 aa, Artificial Sequence
  Valpha7.2/EGFR Heavy Fab-Fab chain
EVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTAPPAPAPELLGGPAAPPAPAPAGGQVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSC

SEQ ID NO: 23 - protein, 476 aa, Artificial Sequence
  EGFR/Valpha7.2 Fab-Fab Heavy chain
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALCLVKDYFPEPVTVSWNSGALTSGVHTFPAVLQSSGLYSLSSVVDVPSSSLGTQTYICNVNHKPSNTKVDKRVEPKSCDKTHTAPPAPAPELLGGPAAPPAPAPAGGEVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSC

SEQ ID NO: 24 - protein, 703 aa, Artificial Sequence
  EGFR/Va7.2 BiXAb Heavy chain
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALCLVKDYFPEPVTVSWNSGALTSGVHTFPAVLQSSGLYSLSSVVDVPSSSLGTQTYICNVNHKPSNTKVDKRVEPKSCDKTHTAPPAPAPELLGGPAAPPAPAPAGGEVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 704 aa, Artificial Sequence
  Va7.2/EGFR Heavy chain BiXAb chain
EVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTAPPAPAPELLGGPAAPPAPAPAGGQVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Artificial Sequence
  HER2 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 476 aa, Artificial Sequence
  Va7.2/ HER2 Heavy Fab-Fab chain
EVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTAPPAPAPELLGGPAAPPAPAPAGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSC

SEQ ID NO: 28 - protein, 476 aa, Artificial Sequence
  HER2 /Va7.2 Fab-Fab Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSCDKTHTAPPAPAPELLGGPAAPPAPAPAGGEVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSC

SEQ ID NO: 29 - protein, 703 aa, Artificial Sequence
  Va7.2/ HER2 Heavy chain BiXAb chain
EVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTAPPAPAPELLGGPAAPPAPAPAGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 703 aa, Artificial Sequence
  HER2/Va7.2 BiXAb Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKRVEPKSCDKTHTAPPAPAPELLGGPAAPPAPAPAGGEVQLQQSGAELVKPGASVKLSCTASGFNIKDTHMHWVKQRPEQGLEWIGRTDPASGDTKYDPKFQGKATITADTSSNTAYLHLSSLTSEDTAVYYCAHYYRDDVNYAMDYWGQGTTVTVSSASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK